SEQUENCE LISTING (20 sequences)

SEQ ID NO: 1 - DNA, 21 bp, Artificial
  Synthetic
cgaactctttcggtcgtaccc

SEQ ID NO: 2 - DNA, 22 bp, Artificial
  Synthetic
cgaatctgcaccgtagttgagc

SEQ ID NO: 3 - DNA, 21 bp, Artificial
  Synthetic
cagaccagcctgacagatttc

SEQ ID NO: 4 - DNA, 22 bp, Artificial
  Synthetic
tcctgacccacagcagaagagg

SEQ ID NO: 5 - DNA, 30 bp, Artificial
  Synthetic
cgccgtgcacagggtgtcacgttgcaagac

SEQ ID NO: 6 - DNA, 24 bp, Artificial
  Synthetic
caagccaaccacggcctccagaag

SEQ ID NO: 7 - DNA, 27 bp, Artificial
  Synthetic
agcagcaatgactgtgatgcacttgag

SEQ ID NO: 8 - DNA, 27 bp, Artificial
  Synthetic
gctctccagcattcgtcagtccatccc

SEQ ID NO: 9 - DNA, 24 bp, Artificial
  Synthetic
cgctgttctcctcttcctcatctc

SEQ ID NO: 10 - DNA, 24 bp, Artificial
  Synthetic
ccagtcatagccgaatagcctctc

SEQ ID NO: 11 - DNA, 23 bp, Artificial
  Synthetic
gcgtaccgctaagggttcaaaac

SEQ ID NO: 12 - DNA, 23 bp, Artificial
  Synthetic
tcctcacagtgaccaagaacctg

SEQ ID NO: 13 - DNA, 23 bp, Artificial
  Synthetic
ctgctcaactacggtgcagattc

SEQ ID NO: 14 - DNA, 25 bp, Artificial
  Synthetic
gcaaatatcgcacgatgtcttgatg

SEQ ID NO: 15 - DNA, 24 bp, Artificial
  Synthetic
cgctgttctcctcttcctcatctc

SEQ ID NO: 16 - DNA, 24 bp, Artificial
  Synthetic
ccagtcatagccgaatagcctctc

SEQ ID NO: 17 - DNA, 21 bp, Artificial
  Synthetic
cgaactctttcggtcgtaccc

SEQ ID NO: 18 - DNA, 22 bp, Artificial
  Synthetic
cgaatctgcaccgtagttgagc

SEQ ID NO: 19 - DNA, 20 bp, Artificial
  Synthetic
cgatcctcccgaattgacta

SEQ ID NO: 20 - DNA, 22 bp, Artificial
  Synthetic
gcacattctgcactacaactcc

(19) United States
(12) Patent Application Publication
(10) Pub. No.: US 2008/0187917 A1
(43) Pub. Date: Aug. 7, 2008

(54) Compositions and methods for repressing the Ink4a and Arf senescence pathways

(75) Inventors: Sean Morrison, Ann Arbor, MI (US); Anna Molofsky, San Francisco, CA (US); Ricardo Pardal, Sevilla (ES)

Correspondence Address: Casimir Jones, S.C., 440 Science Drive, Suite 203, Madison, WI, 53711, US

(73) Assignee: The Regents of the University of Michigan, Ann Arbor, MI (US)

(21) Appl. No.: 11/709,623

(22) Filed: Feb. 22, 2007

Related U.S. Application Data
(60) Provisional application No. 60/775,471, filed on Feb. 22, 2006.

(51) Int. Cl. C12Q 1/68; C12N 5/02

(52) U.S. Cl. 435/6; 435/375

(57) ABSTRACT

The present invention relates to methods and compositions for regulating cell growth and development. In particular, the present invention relates to methods and compositions for repressing Ink4a and Arf pathways.

This application claims priority to provisional application Ser. No. 60/775,471, filed Feb. 22, 2006, which is herein incorporated by reference in its entirety.

This Application was supported in part by NIH Grant No. R01 NS40750. The government may have certain rights in the invention.

### FIELD OF THE INVENTION

The present invention relates to methods and compositions for regulating cell growth and development. In particular, the present invention relates to methods and compositions for promoting the regenerative capacity of cells by repressing Ink4a and Arf pathways.

### BACKGROUND OF THE INVENTION

Recent published reports on the isolation and successful culturing of the first human pluripotent stem cell lines have generated great excitement and have brought biomedical research to the edge of a new frontier (National Institutes of Health, Office of the Director, “Stem Cells: A Primer”). Stem cells have the ability to divide for indefinite periods in culture and to give rise to specialized cells.

The pluripotent stem cells undergo further specialization into stem cells that are committed to give rise to cells that have a particular function. Examples of this include blood stem cells, which give rise to red blood cells, white blood cells and platelets; and skin stem cells that give rise to the various types of skin cells. These more specialized stem cells are called multipotent.

While stem cells are extraordinarily important in early human development, multipotent stem cells are also found in children and adults. For example, consider one of the best understood stem cells, the blood stem cell. Blood stem cells reside in the bone marrow of every child and adult, and in fact, they can be found in very small numbers circulating in the blood stream. Blood stem cells perform the critical role of continually replenishing our supply of blood cells—red blood cells, white blood cells, and platelets—throughout life. A person cannot survive without blood stem cells.

Multipotent stem cells have not been found for all types of adult tissue, but discoveries in this area of research are increasing. For example, until recently, it was thought that stem cells were not present in the adult nervous system, but, in recent years, neural stem cells have been isolated from the rat and mouse nervous systems. The experience in humans is more limited. In humans, neural stem cells have been isolated from fetal tissue and a kind of cell that may be a neural stem cell has been isolated from adult brain tissue that was surgically removed for the treatment of epilepsy.

Research on human adult stem cells suggests that these multipotent cells have great potential for use in both research and in the development of cell therapies. For example, there would be many advantages to using adult stem cells for transplantation. If one can isolate the adult stem cells from a patient, coax them to divide and direct their specialization and then transplant them back into the patient, it is unlikely that such cells would be rejected by the patient's immune system. The use of adult stem cells for such cell therapies could reduce the practice of using stem cells that were derived from human embryos or human fetal tissue, sources that can be practically and ethically problematic.

Thus, what is needed are methods of manipulating stem cells to provide improved treatment methods and improved methods for isolating and analyzing stem cells (e.g., for drug screening applications).

### SUMMARY OF THE INVENTION

The present invention relates to methods and compositions for regulating cell growth and development. In particular, the present invention relates to methods and compositions for promoting the regenerative capacity of cells by repressing Ink4a and Arf pathways. Accordingly, in some embodiments, the present invention provides methods of inhibiting cell senescence pathways by inhibiting the function of Ink4a and Arf. The present invention also provides methods of screening compounds for the ability to inhibit Ink4a and Arf function. Such compounds find use, for example, in the treatment and analysis of diseases related to aging, such as degenerative diseases as well as preventing or reducing age-related declines in neural regenerative activity.

For example, in some embodiments, the present invention provides a method of enhancing stem cell function comprising inhibiting the function of a gene such as Arf and/or Ink4a in a cell (e.g., a neural stem cell). In some embodiments, the cell lacks a functional Bmi-1 gene. In some embodiments, the cell is located in vitro, ex vivo, or in vivo. In some embodiments, inhibiting the function of a gene comprises contacting the cell with a small molecule inhibitor, siRNA or antisense oligonucleotide complementary to the gene, and the like. In some embodiments, the inhibitor directly inhibits Ink4a activity by interacting with Ink4a protein or altering Ink4a protein expression. In other embodiments, Ink4a is inhibited by regulating signaling pathways that alter Ink4a activity. In some embodiments, the cell is in a subject having symptoms of a neurodegenerative disease. In preferred embodiments, inhibiting the function of a gene reduces symptoms of the neurodegenerative disease.

The present invention further provides a method of screening compounds, comprising contacting a neural stem cell that expresses a gene selected from the group consisting of Arf and Ink4a with a test compound; and comparing the level of proliferation of the cell in the presence of the test compound with the level in the absence of the test compound or simply observing the effect of the compound without reference to a control sample. In some embodiments, the test compound is a small molecule that inhibits the function of the polypeptide encoded by the gene (e.g., inhibits Ink4a activity). In some embodiments, the test compound is an siRNA or an antisense oligonucleotide complementary to the gene. In some embodiments, the test compound inhibits the expression of the gene. In other embodiments, the test compound inhibits the function of Arf or Ink4a (e.g., inhibits p16-Ink4a function). In some embodiments, the cell is in vitro, ex vivo, or in vivo. In certain embodiments, the cell is in a non-human animal (e.g., a non-human animal lacking a functional Bmi gene). In some embodiments, the cell is a stem cell (e.g., a neural stem cell). In other embodiments, the test compound increases Ink4a expression in the neural stem cell. In some embodiments, the compound promotes regeneration after injury or disease. In other embodiments, the test compound promotes regeneration in aging tissues.

In other embodiments, the present invention provides a method of screening compounds, comprising contacting a stem cell that expresses a gene (e.g., Arf or Ink4a) with a test compound; and comparing the level of expression of the gene in the presence of the test compound with the level in the absence of the test compound. In preferred embodiments, the level of expression of the gene is increased in the presence of the test compound. In some embodiments, the test compound inhibits the growth of a cancer cell. In other embodiments, the test compound kills cancer cells.

Other embodiments of the invention are described in the description and examples below.

### DESCRIPTION OF THE FIGURES

FIG. 1 shows that Ink4a deficiency significantly increased neural stem cell frequency in Bmi-1−/− mice based on in vitro and in vivo assays. A) Ink4a deficiency partially rescued the percentage of SVZ cells from Bmi-1−/− mice that formed multipotent neurospheres in culture. * indicates significantly different (P<0.05 by T-test) from Bmi-1+/+Ink4a+/+, # indicates significantly different from Bmi-1−/− Ink4a+/+, and § indicates significantly different from Bmi-1+/+Ink4a−/−. B) Ink4a deficiency also partially rescued the frequency of adult gut NCSCs from Bmi-1−/− mice that formed multipotent neurospheres in culture. C) Ink4a deficiency partially rescued the frequency of p75+ cells. D) p75+NCSC frequency (boxed region of each plot) among freshly dissociated gut wall cells was reduced by Bmi-1 deficiency, and increased by Ink4a deficiency.

FIG. 2 shows that Ink4a deficiency partially rescued SVZ proliferation and gut neurogenesis in Bmi-1−/− mice, but not cerebellum development or overall growth or survival. A) The rate of proliferation in the SVZ of Bmi-1−/− mice was significantly increased by Ink4a deficiency. * indicates significantly different (p<0.05 by T-test) from Bmi-1+/+Ink4a+/+, and # indicates significantly different from Bmi-1−/− Ink4a+/+. B) Ink4a deficiency partially rescued the number of myenteric plexus neurons per cross-section through the distal small intestine of Bmi-1−/− mice. C-E) Hematoxylin and eosin stained saggital sections of adult cerebellum demonstrate that Ink4a deficiency did not affect cerebellum development in Bmi-1−/− mice (mean ±SD for 4 mice per genotype and 7 to 25 measurements per mouse). F) Ink4a deficiency did not significantly affect the growth of either Bmi-1+/+ or Bmi-1−/− mice, though Bmi-1−/− mice were significantly (p<0.05) smaller than Bmi-1+/+mice at all time points. (G) Bmi-1−/− mice were born at nearly normal frequencies but usually died prior to postnatal day 30.

FIG. 3 shows p19Arf expression increased in the absence of Bmi-1 and Arf deficiency significantly increased the frequency and self-renewal of neural stem cells from the SVZ and gut of Bmi-1−/− mice. A) p19Arf levels were increased in uncultured SVZ and cerebellum of 4 week old Bmi-1−/− mice. B) p19Arf was increased in Bmi-1−/− CNS neurospheres cultured for 13 days. C) Arf deficiency significantly increased self-renewal (the number of secondary neurospheres generated per subcloned primary neurosphere) within Bmi-1+/+ and Bmi-1−/− CNS neurospheres. D) Arf deficiency significantly increased the percentage of cells from the Bmi-1−/− SVZ that formed multipotent CNS neurospheres. E) Arf deficiency significantly increased self-renewal within Bmi-1+/+ and Bmi-1−/− PNS neurospheres. F) Arf deficiency significantly increased the percentage of cells from the Bmi-1−/− outer gut wall that formed multipotent PNS neurospheres (4 mice per genotype, in 4 independent experiments).

FIG. 4 shows that Arf deficiency partially rescued neural development, but not overall growth or survival in Bmi-1-deficient mice. A) The rate of proliferation (% BrdU+ cells) in the SVZ of 4 to 8 week old Bmi-1−/− (but not Bmi-1+/+) mice was significantly increased by Arf deficiency. * indicates significantly different (p<0.05 by T-test) from Bmi-1+/+Arf+/+, # indicates significantly different from Bmi-1−/−Arf+/+, and § indicates significantly different from Bmi-1-+/+Arf−/−. B) Arf deficiency increased the number of myenteric plexus neurons per cross-section through the ileum of 4 to 9 week old Bmi-1−/− mice. C-E) Arf deficiency also partially rescued cerebellum growth in 4 to 8 week old Bmi-1−/− mice. F) Arf deficiency did not affect the overall growth of Bmi-1+/+ or Bmi-1−/− mice. (G) The survival of Bmi-1−/− mice was not significantly increased by Arf deficiency (arrows; p=0.552 by logistic regression analysis).

FIG. 5 shows that combined Ink4a-Arf deficiency partially rescued the self-renewal and frequency of neural stem cells from the SVZ and gut of Bmi-1−/− mice. A) Ink4a-Arf deficiency significantly increased self-renewal within Bmi-1+/+ and Bmi-1−/− CNS neurospheres cultured from 4 to 8 week old mice. B) Ink4a-Arf deficiency significantly increased the frequency of cells from the Bmi-1−/− adult SVZ that formed multipotent CNS neurospheres in culture (2 to 7 mice/genotype, studied in 8 independent experiments). C) Ink4a-Arf deficiency significantly increased self-renewal within Bmi-1+/+ and Bmi-1−/− PNS neurospheres cultured from the guts of 4 to 8 week old mice. D) Ink4a-Arf deficiency significantly increased the frequency of cells from the Bmi-1-outer gut wall that formed multipotent PNS neurospheres (3 to 8 mice per genotype, studied in 3 independent experiments).

FIG. 6 shows that combined Ink4a-Arf deficiency partially rescued neural development but not overall growth or survival in adult Bmi-1−/− mice. B) Ink4a-Arf deficiency partially rescued the number of myenteric plexus neurons per cross-section through the distal small intestine of Bmi-1−/− mice. C-E) Ink4a-Arf deficiency also partially rescued cerebellum growth in Bmi-1−/− mice. F) Ink4a-Arf deficiency did not increase the overall growth of either Bmi-1+/+ or Bmi-1−/− mice, though Bmi-1−/− mice were significantly (p<0.01) smaller than Bmi-1+/+mice at all time points after P20. (G) The survival of Bmi-1−/− mice was not increased by Ink4a-Arf deficiency.

FIG. 7 shows that Bmi-1 deficiency significantly reduced the diameter of neurospheres, but this effect was partially rescued by Ink4a, Arf, or Ink4a-Arf deficiency. A) Ink4a deficiency significantly increased the diameter of Bmi-1−/− and Bmi-1+/+neurospheres, consistent with a partial rescue of the proliferation defect within Bmi-1−/− neurospheres. * indicates significantly different (P<0.05 by T-test) from Bmi-1+/+Ink4a+/+, # indicates significantly different from Bmi-1−/−Ink4a+/+, and § indicates significantly different from Bmi-1+/+Ink4a−/−. B) Arf deficiency also significantly increased the diameter of Bmi-1−/− and Bmi-1+/+neurospheres, consistent with a partial rescue of the proliferation defect within Bmi-1−/− neurospheres. C) Ink4a-Arf deficiency significantly increased the diameter of Bmi-1−/− and Bmi-1+/+neurospheres in a way that was consistent with a partial rescue of the proliferation defect within Bmi-1−/− neurospheres. D) Representative photographs of neurospheres cultured from the progeny of Bmi-1+/−Ink4a-Arf+/− matings.

FIG. 8 shows that p16Ink4a is expressed in the cerebellum of adult Bmi-1−/− mice.

FIG. 9 shows that the brain masses of adult Bmi-1−/− mice were significantly reduced relative to littermate controls regardless of Ink4a and/or Arf deletion. A) Brain masses in adult (all mice were 4 to 8 weeks old) Bmi-1−/−Ink4a+/+mice were significantly reduced relative to Bmi-1+/+Ink4a+/+controls (*, p<0.01) and Ink4a deficiency had no detectable effect on brain mass. B) Brain masses in Bmi-1−/−Arf+/+ mice were also significantly smaller than Bmi-1+/+Arf+/+controls. C) Brain masses in Bmi-1−/−Ink4a-Arf+/+ mice were significantly smaller than in Bmi-1+/+Ink4a-Arf+/+ mice.

FIG. 10 shows that cip1 expression increases in the absence of Bmi-1 and decreases in the absence of Arf in CNS (A; 4 samples per genotype cultured for 10-11 days) and PNS (B; 2-3 samples per genotype, cultured for 12-14 days) neurospheres.

FIG. 11 shows that Bmi-1 deficiency significantly reduced the percentage of cells within primary neurospheres capable of forming secondary neurospheres, but Ink4a, Arf, or Ink4a-Arf deficiency significantly increased this percentage. A) Bmi-1+/−Ink4a+/− matings (mean ±SD for 2 mice per genotype in two independent experiments, 6 neurospheres per mouse). B) Bmi-1+/−Arf+/− matings (mean ±SD for 2-4 mice per genotype in 4 independent experiments, 4-6 neurospheres per mouse). C) Bmi-1+/−Ink4a-Arf+/− matings (mean ±SD for 2-5 mice per genotype in 4 independent experiments, 4-6 neurospheres per mouse).

FIG. 12 shows that neural progenitor function declines with age. The percentage of cells from the SVZ that form multipotent neurospheres in culture declines with age (A; *, p<0.05 relative to P60; 3 independent experiments; 5-6 mice per age). The self-renewal potential of these primary neurospheres also declines with age (B; *, p<0.05; 3 independent experiments; 5-6 mice per age). The rate of proliferation in the SVZ (% BrdU+ cells after a two hour pulse) also declines significantly with age (C, D; 3 mice per age; 5-7 sections counted per mouse). Ink4a expression increased with age by quantitative (real-time) PCR in uncultured SVZ cells (E; 3 independent samples per age).

FIG. 13 shows that Ink4a is partially responsible for the age-related declines in stem and progenitor cell function in the SVZ. A) The percentage of SVZ cells that formed multipotent neurospheres in culture was significantly reduced in old wild-type mice as compared to young mice (*, p<0.01) but significantly increased in old mice by Ink4a deficiency (#, p<0.01 relative to old wild-type). B) Self-renewal potential (the number of secondary neurospheres generated per subcloned primary neurosphere) was also significantly reduced in CNS neurospheres from old wild-type mice as compared to young mice (* p<0.05). C) The percentage of SVZ cells that incorporated a two hour pulse of BrdU was significantly reduced in old mice as compared to young mice (* p<0.01) but significantly increased in old mice by Ink4a deficiency (#, p<0.01 relative to old wild-type by paired T-test). The percentage of proliferating cells in old Ink4a-deficient mice was still significantly less than in young mice (* p<0.01) and coronal sections through the lateral ventricle showed cortical atrophy, marked by enlarged ventricles (*), in both wild-type and Ink4a deficient old mice (D).

FIG. 14 shows that Ink4a causes age-related declines in olfactory bulb neurogenesis. Panels a-d show low magnification images (scale bar=20 um) from saggital sections of the olfactory bulb of old wild type (a,b; same field of view) and Ink4a-deficient (c,d; same field of view) mice. Panels e-h show higher magnification images (scale bar=10 um) from one field of view from an old Ink4a-deficient mouse: BrdU+NeuN+ neuron (arrow) and BrdU+NeuN− non-neuronal cell (arrowhead). Panel h is a three-dimensional, reconstructed side view (80° turn in the z-axis) of panel g. Neurogenesis significantly (*, p<0.05) declined with age (i; BrdU+NeuN+neurons as a percentage of all NeuN+neurons). The frequency of BrdU+NeuN− non-neuronal cells was not significantly affected by Ink4a deficiency (j; also as a percentage of NeuN+neurons).

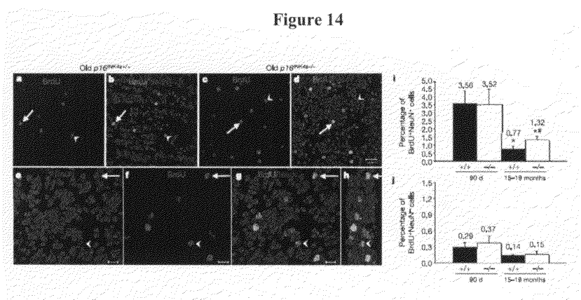

FIG. 15 shows that Ink4a does not significantly affect proliferation or neurogenesis in the dentate gyrus of old mice. Panels a, c, and e show one field of view from the dentate gyrus of an old wild type mouse while panels b, d, and f show one field of view from the dentate gyrus of an old Ink4a-deficient mouse. NeuN (a,b,e,f) labels neurons in the dentate gyrus while BrdU (c,d,e,f) labels cells that were born during the BrdU pulse (8 days for the analysis of proliferation in the subgranular layer (g) or 8 days followed by a 4 week chase without BrdU for the analysis of neurogenesis in the granular layer (a-f; h)). A BrdU+NeuN+neuron is shown in panels a,c,e (arrow). A BrdU+NeuN− non-neuronal cell is shown in panels b,d,f (arrowhead). In the proliferation analysis, the number of BrdU+ cells per mm of subgranular layer (SGL) in old mice was not affected by Ink4a-deficiency (g). The numbers of BrdU+NeuN− non-neuronal cells and BrdU+NeuN+ neurons per mm of dentate gyrus (DG) in old mice were also not significantly affected by Ink4a deficiency (h).

FIG. 16 shows that neural crest stem cell frequency decreases, and p16Ink4a expression increases with age in the gut but Ink4a deficiency does not rescue stem cell frequency. a; data are from four independent experiments. b; data are from four independent experiments. p16Ink4a expression increased with age in p75+ gut cells by quantitative PCR (c), and by Western blot of gut wall cells (myenteric plexus plus muscle layer) (d). Ink4a deficiency did not affect the frequency of cells from the gut wall that formed multilineage neural crest stem cell colonies in culture (e; *, p<0.05 relative to young wild type mice; data are from four independent experiments).

### DEFINITIONS

To facilitate an understanding of the present invention, a number of terms and phrases are defined below:

The term “transplant” refers to tissue used in grafting, implanting, or transplanting, as well as the transfer of tissues from one part of the body to another, or the transfer of tissues from one individual to another, or the introduction of biocompatible materials into or onto the body. The term “transplantation” refers to the grafting of tissues from one part of the body to another part, or to another individual.

As used herein, the term “stem cell” refers to self-renewing multipotent cells that are capable of giving rise to more stem cells, as well as to various types of terminally differentiated cells.

As used herein, the term “host” refers to any warm blooded mammal, including, but not limited to, humans, non-human primates, rodents, and the like. Typically, the terms “host” and “patient” are used interchangeably herein in reference to a human subject.

As used herein, the terms “defective tissues” and “defective cells” refer to tissues and cells that are marked by subnormal structure, function, or behavior. Defects responsible for the defective tissues and cells include known or detectable defects, as well as, unknown or undetectable defects.

As used herein, the term “neural defect” or “neurological disorder” refers to a defect involving or relating to the nervous system (including central and peripheral nervous systems). Some neural defects are caused by injury to the nervous system or defective tissues or cells of the nervous system, while other defects are caused by injury to cells that affect the nervous system or defective tissues or cells that affect the nervous system. As used herein, the term “neurally defective mammal” refers to a mammal having one or more neural defects. When a neural defect is “ameliorated,” the condition of the host is improved. For example, amelioration can occur when defective tissue is returned partially or entirely to a normal state. However, amelioration can also occur when tissue remains subnormal, but is otherwise altered to benefit the host.

As used herein, the term “degenerative disease” refers to a disease or disorder characterized by a decrease in function or degradation of normally functional tissues or organs.

As used herein, the term “non-human animals” refers to all non-human animals. Such non-human animals include, but are not limited to, vertebrates such as rodents, non-human primates, ovines, bovines, ruminants, lagomorphs, porcines, caprines, equines, canines, felines, aves, etc.

The term “biologically active,” as used herein, refers to a protein or other biologically active molecules (e.g., catalytic RNA) having structural, regulatory, or biochemical functions of a naturally occurring molecule.

The term “agonist,” as used herein, refers to a molecule which, when interacting with a biologically active molecule, causes a change (e.g., enhancement) in the biologically active molecule, which modulates the activity of the biologically active molecule. Agonists may include proteins, nucleic acids, carbohydrates, small molecule drugs or any other molecules that bind or interact with biologically active molecules.

The terms “antagonist” or “inhibitor,” as used herein, refer to a molecule which, when interacting with a biologically active molecule, blocks or reduces the biological activity of the biologically active molecule. Antagonists and inhibitors may include proteins, nucleic acids, carbohydrates, small molecule drugs or any other molecules that bind or interact with biologically active molecules. Inhibitors and antagonists can affect the biology of entire cells, organs, or organisms (e.g., an inhibitor that slows tumor growth).

The term “modulate,” as used herein, refers to a change in the biological activity of a biologically active molecule. Modulation can be an increase or a decrease in activity, a change in binding characteristics, or any other change in the biological, functional, or immunological properties of biologically active molecules.

The term “isolated” when used in relation to a cell, as in “an isolated cell” or “isolated cells” refers to cells that are separated and enriched in a sample so as to remove the isolated cell(s) from other cells with which it is ordinarily associated in its natural environment. For example, isolated stem cells are stem cells that are removed from their natural environment and enriched in a sample, such that the sample housing the stem cells contains a higher percentage of stem cells than a corresponding sample found in a tissue in its natural environment.

The term “transgene” as used herein refers to a foreign gene that is placed into an organism or cell by, for example, transfection or introducing the foreign gene into newly fertilized eggs or early embryos. The term “foreign gene” refers to any nucleic acid (e.g., gene sequence) that is introduced into the genome of an animal or cell by experimental manipulations and may include gene sequences found in that animal so long as the introduced gene does not reside in the same location as does the naturally-occurring gene.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell which has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “test compound” refers to any molecule, chemical entity, pharmaceutical, drug, and the like that can be used to treat or prevent a disease, illness, sickness, or disorder of bodily function. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue (e.g., tissues of the gut or central nervous system), liquid foods (e.g., milk), and solid foods (e.g., vegetables).

As used herein, the term “immunoglobulin” or “antibody” refer to proteins that bind a specific antigen. Immunoglobulins include, but are not limited to, polyclonal, monoclonal, chimeric, and humanized antibodies, Fab fragments, F(ab′)₂ fragments, and includes immunoglobulins of the following classes: IgG, IgA, IgM, IgD, IgE, and secreted immunoglobulins (sIg). Immunoglobulins generally comprise two identical heavy chains and two light chains. However, the terms “antibody” and “immunoglobulin” also encompass single chain antibodies and two chain antibodies.

As used herein, the term “antigen binding protein” refers to proteins that bind to a specific antigen. “Antigen binding proteins” include, but are not limited to, immunoglobulins, including polyclonal, monoclonal, chimeric, and humanized antibodies; Fab fragments, F(ab′)₂ fragments, and Fab expression libraries; and single chain antibodies.

The term “epitope” as used herein refers to that portion of an antigen that makes contact with a particular immunoglobulin.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “transgene” refers to a heterologous gene that is integrated into the genome of an organism (e.g., a non-human animal) and that is transmitted to progeny of the organism during sexual reproduction.

As used herein, the term “transgenic organism” refers to an organism (e.g., a non-human animal) that has a transgene integrated into its genome and that transmits the transgene to its progeny during sexual reproduction.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

As used herein, the term “drug” refers to pharmacologically active molecules that are used to diagnose, treat, or prevent diseases or pathological conditions in a physiological system (e.g., a subject, or in vivo, in vitro, or ex vivo cells, tissues, and organs). Drugs act by altering the physiology of a living organism, tissue, cell, or in vitro system to which the drug has been administered.

### DETAILED DESCRIPTION OF THE INVENTION

The present invention relates to methods and compositions for regulating cell growth and development. In particular, the present invention relates to methods and compositions for promoting the regenerative capacity of cells (e.g., restoring progenitor activity in aging tissues) by repressing Ink4a and Arf pathways (e.g., reducing enzyme activity). In some embodiments, the present invention provides methods of treating neurodegenerative diseases or age-related declines in neural regenerative activity by reducing Ink4a expression. In other embodiments, the present invention provides methods of treating cancer by increasing Ink4a expression.

Stem cell self-renewal is intimately linked with stem cell aging (de Haan and Van Zant, Blood 93: 3294-3301, 1999; Chen et al., Exp Hematol 28: 442-50, 2000; Park et al., Clin Invest 113: 175-9, 2004). Stem cells must persist throughout adult life in numerous tissues in order to replace the mature cells that are lost to turnover, injury, or disease. The mechanism by which stem cells persist throughout life involves self-renewal—stem cell mitoses that generate one or two daughter stem cells (Morrison et al., Cell 88: 287-298 1997; Molofsky et al., Curr Opin Cell Biol 16: 700-7, 2004). Stem cells self-renew throughout life in numerous tissues, including the CNS (Maslov et al., J Neurosci 24: 1726-33, 2004), PNS (Kruger et al., Neuron 35: 657-69, 2002), and hematopoietic system (Harrison, Mechanisms of Ageing and Development 9: 409-426, 1979; Morrison et al., Nature Medicine 2: 1011-1016, 1996). However, these stem cells exhibit age-related declines in frequency and function (Chen et al., 2000, supra; Geiger and Van Zant, cells. Nat Immunol 3: 329-33, 2002; Maslov et al., J Neurosci 24: 1726-33, 2004).

The polycomb family transcriptional repressor Bmi-1 is required for the self-renewal and postnatal maintenance of hematopoietic stem cells (Lessard and Sauvageau, Nature 423: 255-260, 2003; Park et al., Nature 423: 302-305, 2003), and neural stem cells from the CNS and PNS (Molofsky et al., Nature 425: 962-7, 2003). In each tissue Bmi-1−/− stem cells form in normal numbers and appear to differentiate normally, but exhibit a postnatal self-renewal defect that leads to their depletion by early adulthood. Yet Bmi-1 is not generically required for the proliferation of all cells (Molofsky et al. 2003, supra). Bmi-1 functions as part of a protein complex that maintains gene silencing by regulating chromatin structure (Valk-Lingbeek et al., Cell 118: 409-18, 2004). Except for a mild skeletal transformation, Bmi-1−/− mice are normal in size and appearance at birth (van der Lugt et al., Mechanisms of Development 58: 153-164, 1994). However, they exhibit progressive postnatal growth retardation and die by early adulthood with signs of hematopoietic failure (hypocellular bone marrow) and neurological abnormalities (seizures and ataxia) (van der Lugt et al. 1994, supra).

Bmi-1−/− mice develop several specific neural abnormalities. The rate of proliferation in the forebrain subventricular zone (where CNS stem cells undergo neurogenesis), is reduced by postnatal day 30 (P30) when stem cell depletion in this region becomes severe (Molofsky et al. 2003, supra). The cerebellum also fails to develop normally, partly because Bmi-1 is required for the proliferation of granule precursor cells (Leung et al., Nature 428: 337-41, 2004). Finally, adult Bmi-1−/− mice exhibit fewer neurons in the myenteric plexus of the gut as the neural crest stem cells (NCSCs) in this region of the postnatal PNS become depleted (Molofsky et al. 2003, supra). These defects indicate that the pathways regulated by Bmi-1 have important consequences for neural development.

Bmi-1 directly or indirectly represses transcription at the Ink4a-Arf locus (Jacobs et al., Nature 397: 164-168; 1999a; Jacobs et al., Genes and Development 13: 2678-2690, 1999b), which encodes two inhibitors of cell proliferation (Sherr, Nature Reviews Molecular Cell Biology 2: 731-737, 2001).

Ink4a encodes p16Ink4a, a cyclin-dependent kinase inhibitor that promotes Rb activation. Arf encodes p19Arf, which promotes p53 activation. p16Ink4a and p19Arf are induced in cultured primary cells and can cause these cells to undergo senescence (reviewed in Lowe and Sherr, Current Opinion in Genetics & Development 13: 77-83, 2003). Bmi-1 over-expression can prevent senescence and extend the replicative lifespan of primary cells by reducing p16Ink4a and p19Arf expression (Jacobs et al. 1999a, supra; Dimri et al., Cancer Res 62: 4736-45, 2002; Itahana et al., Molecular and Cellular Biology 23: 389-401, 2003). Deletion of the Ink4a-Arf locus from Bmi-1−/− mice rescues the ability of mouse embryonic fibroblasts to proliferate in culture and at least partially rescues defects in cerebellum development (Jacobs et al. 1999a, supra). p16Ink4a expression is elevated in Bmi-1−/− neural stem cells and deletion of Ink4a from Bmi-1−/− mice partially rescues neural stem cell self-renewal in culture (Molofsky et al., 2003, supra). The Ink4a-Arf locus is thus an important mediator of Bmi-1 function.

Ink4a and Arf have been viewed as checkpoint genes that are induced in response to abnormal mitogenic stimuli to prevent uncontrolled proliferation. p16Ink4a and p19Arf often cannot be detected in developing tissues in vivo, but are induced in response to stress and impair cancer cell proliferation (Sherr 2001, supra; Lowe and Sherr 2003, supra; Sharpless et al., Oncogene 23: 379-85, 2004). In addition to this surveillance role, p16Ink4a and p19Arf are upregulated in some tissues during aging (Zindy et al., Oncogene 15: 203-211, 1997; Lowe and Sherr 2003, supra; Krishnamurthy et al., J Clin Invest 114: 1299-307, 2004).

Experiments conducted during the course of development of the present invention utilized mutant mice that lack Bmi-1 (van der Lugt et al. 1994, supra) and/or Ink4a (Sharpless et al., Nature 413: 86-91, 2001), Arf (Kamijo et al., Cell 91: 649-59, 1997), or Ink4a/Arf (Serrano et al., Cell 85: 27-37, 1996). The results indicate that the repression of Ink4a and Arf each represent major methods by which Bmi-1 promotes neural stem cell self-renewal and neural development. This demonstrates that the maintenance of neural stem cells depends upon the repression of senescence pathways that otherwise cause the premature depletion of stem cells. The observation that p16Ink4a is de-repressed in aging neural stem cells indicates that stem cell self renewal and senescence are regulated by a balance between Ink4a-Arf repression and activation.

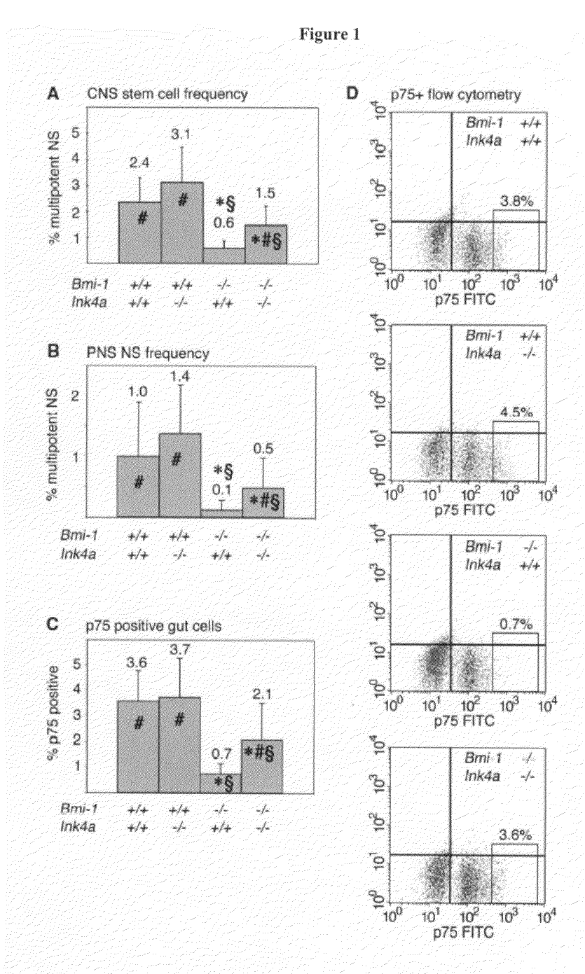

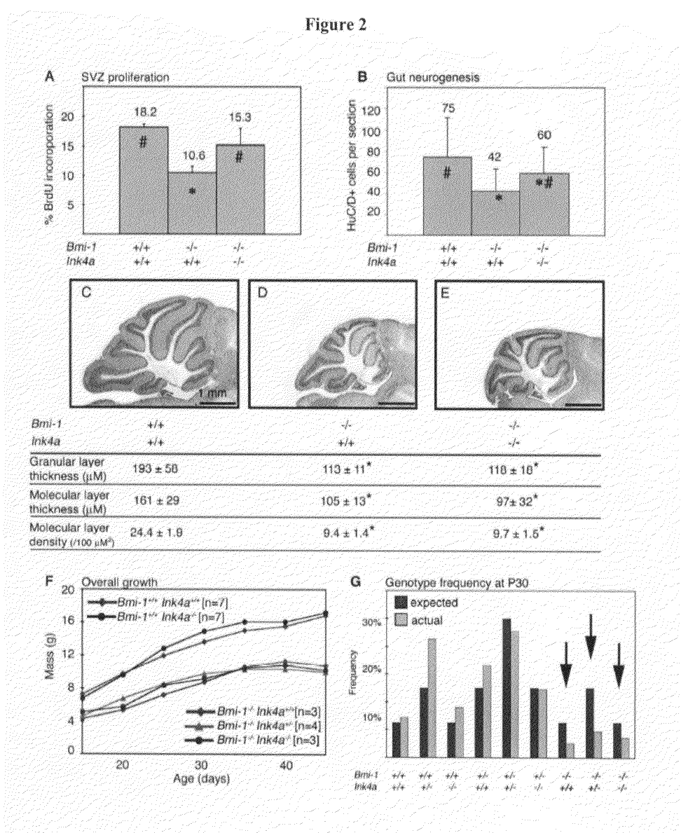

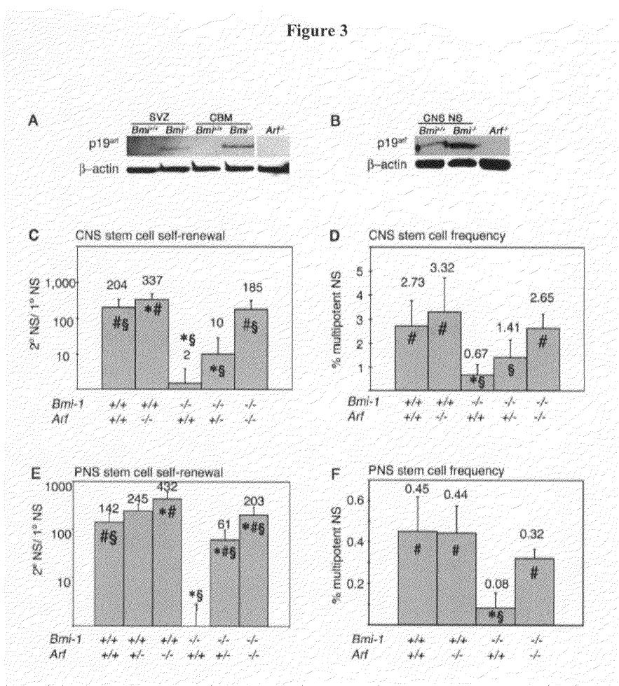

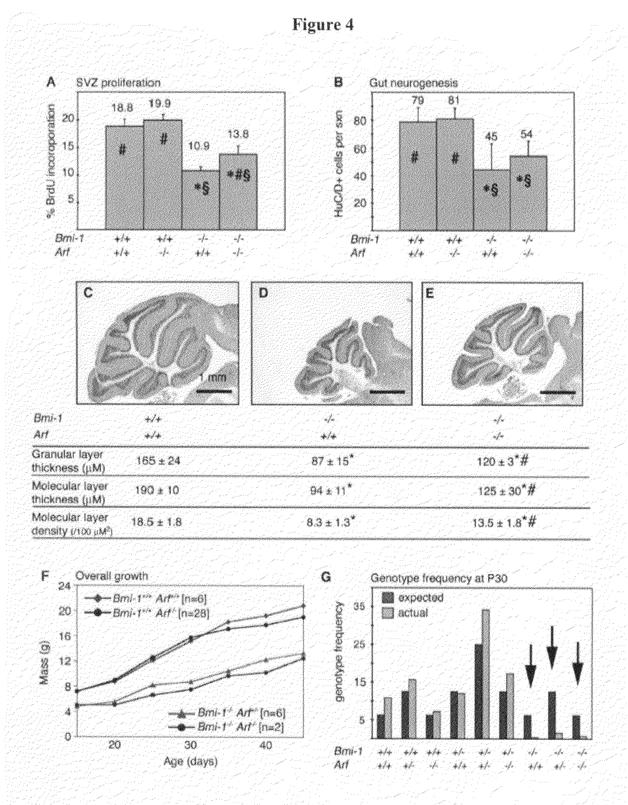

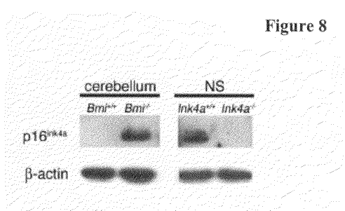

The repression of Ink4a and Arf represent methods by which Bmi-1 promotes postnatal stem cell self-renewal, stem cell maintenance, and development in the nervous system. Ink4a is upregulated postnatally in vitro and in vivo in neural stem cells in the absence of Bmi-1 (Molofsky et al. 2003, supra). Deletion of Ink4a from Bmi-1 deficient mice partially rescued adult neural stem cell self-renewal, and neural stem cell maintenance in the CNS and PNS (FIG. 1). The evidence for the partial rescue of PNS stem cell frequency included both in vitro functional assays and the prospective identification of uncultured p75+NCSCs by flow-cytometry (FIG. 1D). This demonstrated that estimates of stem cell frequency based on the formation of multilineage colonies in culture correlate with independent estimates obtained from the analysis of uncultured cells. p19Arf expression is also elevated in cultured and uncultured Bmi-1−/− neural progenitors (FIG. 3). Like Ink4a, deletion of Arf partially rescued stem cell self-renewal and stem cell frequency in the CNS and PNS of adult Bmi-1−/− mice (FIG. 3). Ink4a and Arf deletion also partially rescued forebrain SVZ proliferation and gut neurogenesis (FIG. 2, 4). However, while Arf deletion partially rescued Bmi-1−/− cerebellum development (FIG. 4C-E), Ink4a deletion did not affect cerebellum development (FIG. 2C-E), despite elevated p16Ink4a expression (FIG. 8). The observation that p16Ink4a impaired proliferation and neurogenesis in the forebrain and gut but not in the cerebellum demonstrates that stem/progenitor cells in different regions of the nervous system differ in their sensitivity to p16Ink4a. Bmi-1−/− neuronal and glial progenitors were insensitive to elevated p16Ink4a expression even while stem cells from the same tissues were strongly inhibited by p16Ink4a (Molofsky et al. 2003, supra). These results demonstrate that differences exist among progenitors within and between tissues in their sensitivity to p16Ink4a.

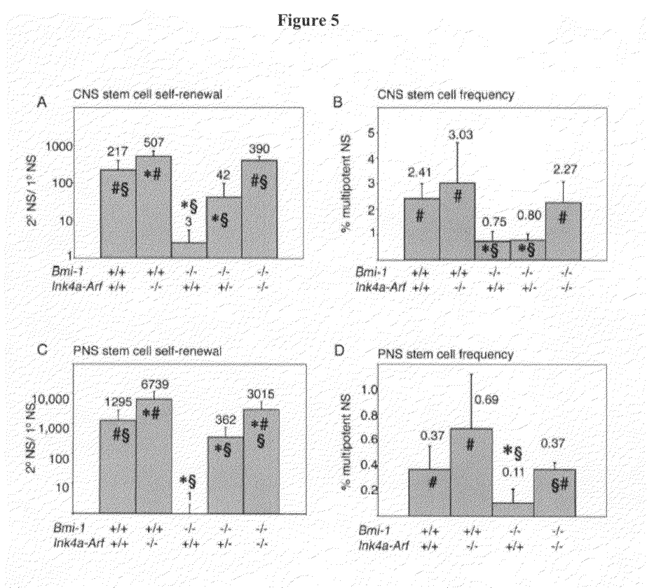

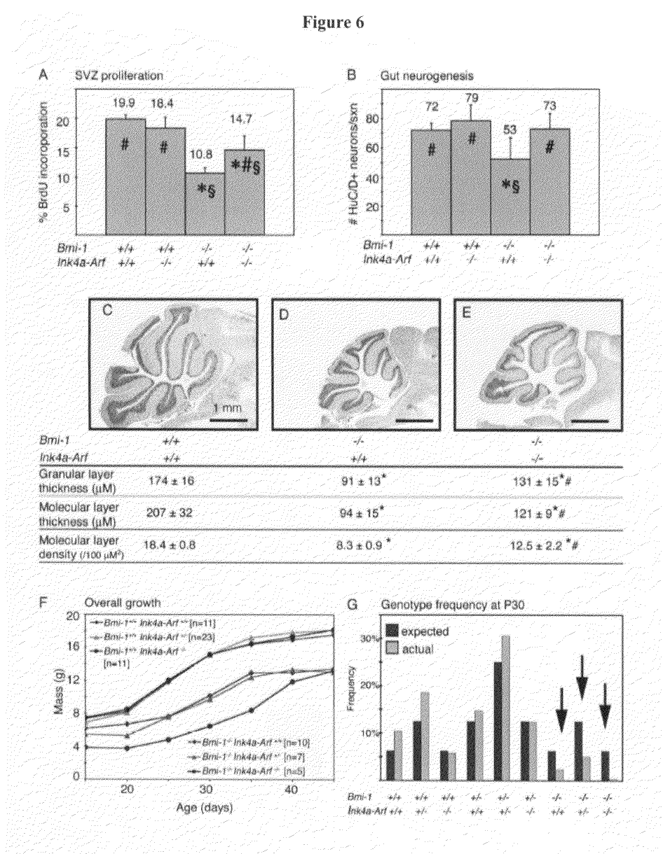

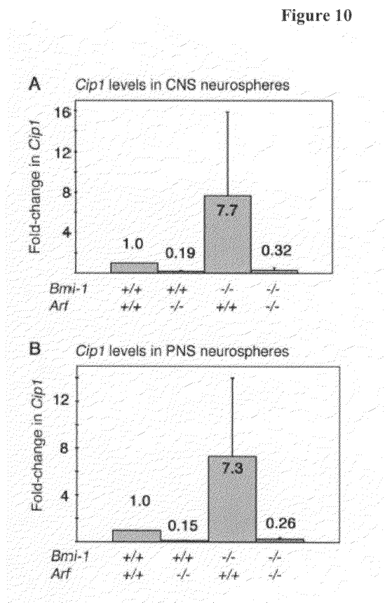

The extent to which Ink4a-Arf deficiency rescued SVZ proliferation (FIG. 6A) was not greater than observed from deletion of Ink4a (FIG. 2A) or Arf alone (FIG. 4A). The extent to which Ink4a-Arf deficiency rescued neural stem cell frequency (FIG. 5B,D) was less than the sum of the effects of Ink4a deficiency (FIG. 1A, 1B, 2B) and Arf deficiency (FIG. 3D, 3F, 4B). This indicates that there is cross regulation between the p16Ink4a and p19Arf pathways. A number of mechanisms by which the p16Ink4a and p19Arf pathways influence each other have been identified (reviewed by Lowe and Sherr 2003, supra). For example, p21cip1 regulates the proliferation/self-renewal of stem cells in the hematopoietic and nervous systems (Cheng et al., Science 287:1804-1808, 2000; Qiu et al., J Exp Med 199: 937-45, 2004). Since p21cip1, like p16Ink4a, promotes Rb activation, changes in p21cip1 expression affect the levels of p16Ink4a that are required to inhibit proliferation (Lowe and Sherr 2003, supra). The present invention is not limited to a particular mechanism. Indeed, an understanding of the mechanism is not necessary to practice the present invention. Nonetheless, it is contemplated that Arf deficiency is predicted to reduce cip1 expression by reducing p53 activity (Lowe and Sherr 2003, supra). Relative to wild-type neurospheres, cip1 RNA levels were 5 to 6 fold reduced in Bmi-1+/+Arf−/− neurospheres, 7-fold increased in Bmi-1−/−Arf+/+neurospheres (as would be predicted based on the increased p19Arf in these cells), and 3 to 4-fold reduced in Bmi-1−/−Arf−/− neurospheres by quantitative PCR (FIG. 10). The present invention is not limited to a particular mechanism. Indeed, an understanding of the mechanism is not necessary to practice the present invention. Nonetheless, it is contemplated that these changes in cip1 levels are a source of cross regulation that could account for the less-than-additive effects of Ink4a and Arf deletion.

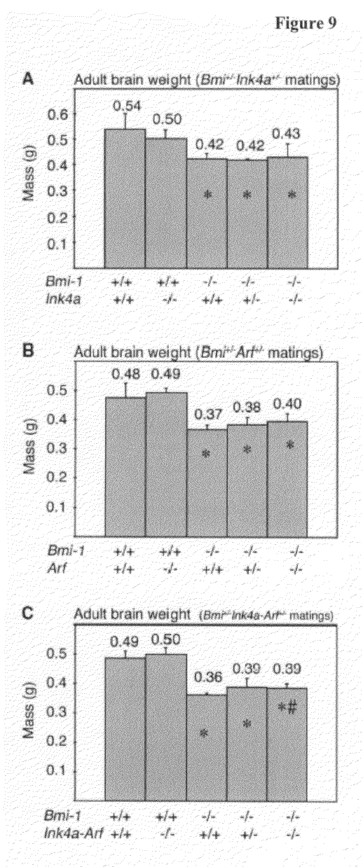

Although deletion of Ink4a and Arf substantially rescued neural development in Bmi-1−/− mice, few if any of these phenotypes were completely rescued. Deletion of Ink4a-Arf had no effect on the overall growth or survival of Bmi-1−/− mice (FIG. 2, 4, 6) and little effect on brain mass (FIG. 9). The fact that Ink4a-Arf deletion substantially rescued CNS stem cell function (FIG. 5) and SVZ proliferation (FIG. 6A), while poorly rescuing overall brain growth (FIG. 9) indicates that it is possible to uncouple the mechanisms by which Bmi-1 regulates stem cell function and tissue growth.

The present invention contemplates additional pathways that function downstream of Bmi-1. Bmi-1 is known to regulate Hox gene expression (Alkema et al., Nature 374: 724-727 1995; van der Lugt et al. 1996, supra; Hanson et al., Proc Natl Acad Sci USA 96: 14372-14377, 1999). Several Hox genes are consistently increased in expression in Bmi-1−/− CNS and PNS neurospheres (Molofsky et al. 2003, supra). A number of other genes have also been observed to increase in expression in Bmi-1−/− neurospheres or bone marrow cells (Molofsky et al. 2003, supra; Park et al., Nature 423: 302-305, 2003), though the functional consequences of these changes in gene expression remain unknown.

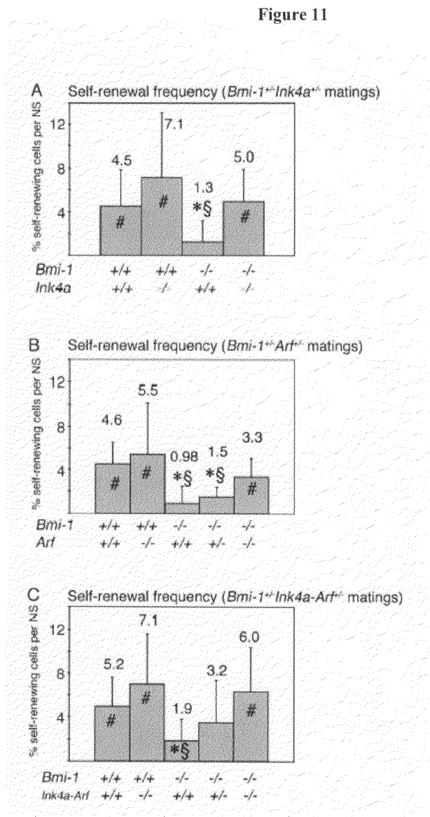

The present invention is not limited to a particular mechanism. Indeed, an understanding of the mechanism is not necessary to practice the present invention. Nonetheless, it is contemplated that Bmi-1 may promote stem cell self-renewal by promoting the maintenance of multipotentiality in addition to promoting proliferation. Bmi-1 promotes hematopoietic stem cell self-renewal by inhibiting differentiation (Iwama et al., Immunity 21: 843-51, 2004). In addition to reducing proliferation within neurospheres, Bmi-1 deficiency also reduced the percentage of cells within neurospheres that remained multipotent (FIG. 11). This provides that Bmi-1 promotes the maintenance of multipotentiality in neural stem cells. p16Ink4a and p19Arf could themselves impair the maintenance of multipotentiality in stem cells by promoting differentiation. The fact that Ink4a or Arf deletion increased the percentage of multipotent cells within Bmi-1−/− neurospheres is also consistent with this concept (FIG. 11).

To the extent that induction of p16Ink4a and p19Arf have been associated with cellular senescence (reviewed by Lowe and Sherr 2003, supra), the data indicate that neural stem cells undergo premature senescence in the absence of Bmi-1 and become depleted by early adulthood. This indicates that the repression of these senescence pathways is a fundamental requirement for the maintenance of stem cells throughout adult life, at least in the nervous system.

While Bmi-1 delays the onset of senescence, it does not completely prevent it. As wildtype mice age they exhibit reduced stem cell frequency and self-renewal potential in both the CNS and PNS. Reduced mitotic activity is also observed in the forebrain SVZ. These age-related changes were associated with increased p16Ink4a expression in NCSCs and SVZ progenitors. All of these phenotypes were also observed in Bmi-1−/− mice, and were partially reversed by Ink4a deletion, indicating that these aging phenotypes are partially caused by the increase in p16Ink4a.

A decline in Bmi-1 expression with age was not observed. This indicates that Bmi-1 represses Ink4a robustly enough to sustain stem cells throughout adult life but not robustly enough to prevent Ink4a expression or stem cell senescence in aging mice. A recent study that observed age-related increases in p16Ink4a expression in other tissues correlated these increases with increased Ets-1 expression (Krishnamurthy et al. 2004, surpa).

The results of experiments conducted during the course of development of the present invention indicated that adult stem cell self-renewal is regulated by a balance between the repression and activation of Ink4a-Arf. These phenomena may reflect the physiological consequences of mechanisms that evolved to balance the need for regenerative capacity in tissues while guarding against uncontrolled proliferation.

Accordingly, in some embodiments, the present invention provides methods of modulating the activity of Ink4a and/or Arf. In some embodiments, the present invention provides compositions and methods of modulating Ink4a and/or Arf in cells, tissues, organs, or individuals with normal, variant, mutant, or deleted Bmi-1 genes. Such methods find use, for example, in the treatment and analysis of degenerative and neurological disorders, in particular, those associated with aging. Such methods find use in the identification of agents (e.g., drugs) that regulate these pathways.

### I. Therapeutic Applications

In some embodiments, the present invention provides therapies for prolonging stem cell function for research and medical applications. In some embodiments, the methods inhibit Arf and/or Ink4a function. In other embodiments, the methods inhibit function by modulating regulators or downstream mediators of Arf and/or Ink4a function. In some embodiments, decreasing Ink4a function is contemplated to promote regenerative capacity in aging tissues. Such methods find use in the treatment of neurodegenerative disease, particularly those associated with ageing.

In other embodiments, the present invention provides methods of treating cancer by upregulating Ink4a expression in cancer cells that express Ink4a or by introducing exogenous Ink4a to cancer cell that do not express Ink4a.

In some embodiments, additional inhibitors of Arf and/or Ink4a function and related pathways are identified using the drug screening applications disclosed herein.

### A. Antisense and RNAi Therapies

In some embodiments, the present invention targets the expression of Arf and/or Ink4a. For example, in some embodiments, the present invention employs compositions comprising oligomeric antisense compounds, particularly oligonucleotides, for use in modulating the function of nucleic acid molecules encoding Arf and/or Ink4a, ultimately modulating the amount of Arf and/or Ink4a protein expressed. This is accomplished by providing antisense compounds (e.g., antisense oligonucleotides, siRNA, etc.) that specifically hybridize with one or more nucleic acids encoding Arf and/or Ink4a. The specific hybridization of an oligomeric compound with its target nucleic acid interferes with the normal function of the nucleic acid.

i. RNA Interference (RNAi)

In some embodiments, RNAi is utilized to inhibit Arf and/or Ink4a function. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC(RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments.

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278: 15991-15997; herein incorporated by reference) describe the use of a type of DNA array called a scanning array to find accessible sites in mRNAs for designing effective siRNAs. These arrays comprise oligonucleotides ranging in size from monomers to a certain maximum, usually Comers, synthesised using a physical barrier (mask) by stepwise addition of each base in the sequence. Thus the arrays represent a full oligonucleotide complement of a region of the target gene. Hybridisation of the target mRNA to these arrays provides an exhaustive accessibility profile of this region of the target mRNA. Such data are useful in the design of antisense oligonucleotides (ranging from 7 mers to 25 mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A1, WO03070966A2, J Mol. Biol. 2005 May 13; 348(4):883-93, J Mol. Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs.

ii. Antisense

In other embodiments, the present invention employs compositions comprising oligomeric antisense compounds, particularly oligonucleotides (e.g., those identified in the drug screening methods described below), for use in modulating the function of nucleic acid molecules encoding Arf and/or Ink4a, ultimately modulating the amount of Arf and/or Ink4a expressed. This is accomplished by providing antisense compounds that specifically hybridize with one or more nucleic acids encoding Arf and/or Ink4a. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of Arf and/or Ink4a. In the context of the present invention, “modulation” means either an increase (stimulation) or a decrease (inhibition) in the expression of a gene. For example, expression may be inhibited to potentially prevent tumor metastasis.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding an Arf and/or Ink4a protein. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in U.S. Patent WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991).

Most preferred embodiments of the invention are oligonucleotides with phosphorothioate backbones and oligonucleosides with heteroatom backbones, and in particular —CH₂, —NH—O—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂—[wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₁ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group. A further preferred modification includes 2′-dimethylaminooxyethoxy (i.e., a O(CH₂)₂ON(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferred modifications include 2′-methoxy(2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2. degree ° C. and are presently preferred base substitutions, even more particularly when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-5-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the antisense oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of the present invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the present invention may be formed as composite structures of two or more oligonucleotides, modified oligonucleotides, oligonucleosides and/or oligonucleotide mimetics as described above.

The present invention also includes pharmaceutical compositions and formulations that include the antisense compounds of the present invention as described below.

### B. Antibody Therapy

In other embodiments, the present invention provides antibodies that target Arf and/or Ink4a or Arf and/or Ink4a signal pathway components. In preferred embodiments, the antibodies used for therapy are humanized antibodies. Methods and compositions for generating antibodies are described below. Exemplary antibodies to Ink4a and Arf are known in the art (See e.g., Murphy et al., Journal of Clinical Pathology 2005; 58:525-534, herein incorporated by reference) and are commercially available (e.g., from Novus Biologicals and Lab Vision).

### C. Small Molecule Drugs

In still further embodiments, the present invention provides drugs (e.g., small molecule drugs) that prevent stem cell senescence and thus promote regenerative capacity in aging tissues by inhibiting the biological activity of Arf and/or Ink4a or altering the biological activity of Arf and/or Ink4a pathway components. In some embodiments, small molecule drugs are identified using the drug screening methods described below. In other embodiments, small molecule drugs are described in WO05073164 and J Biol. Chem. 2004 Jun. 25; 279(26):27187-93, each of which is herein incorporated by reference. Inhibition can be direct or indirect and operate at the protein or gene expression level (see e.g., Amico et al., Cancer Research 2004; 64, 4122-4130, herein incorporated by reference in their entireties).

### D. Genetic and Transplantation Therapies

In yet other embodiments, the present invention contemplates the use of any genetic manipulation for use in modulating the expression of Arf and/or Ink4a. Examples of genetic manipulation include, but are not limited to, delivery of inhibitors of Arf and/or Ink4a (e.g., to cells, tissues, or subjects). Delivery of nucleic acid construct to cells in vitro or in vivo may be conducted using any suitable method. A suitable method is one that introduces the nucleic acid construct into the cell such that the desired event occurs (e.g., expression of an antisense construct). For example, cells may be transfected ex vivo to decrease Arf and/or Ink4a expression and the transfected cells may be transplanted to the site of interest (e.g., nervous system).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

### E. Pharmaceutical Compositions

The present invention further provides pharmaceutical compositions (e.g., comprising the therapeutic compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

### II. Antibodies

The present invention provides isolated antibodies. In preferred embodiments, the present invention provides monoclonal antibodies that specifically bind to an isolated polypeptide comprised of at least five amino acid residues of Arf or Ink4a. These antibodies find use in the diagnostic methods described herein. In some embodiments, antibodies also find use in research applications, drug screening, and therapeutic applications (e.g., antibodies directed to factors that influence Arf and/or Ink4a signaling). In some embodiments, antibodies are expressed in the cell of interest to permit intracellular localization.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against Ink4a or Arf). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against Arf and/or Ink4a) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, Arf and/or Ink4a protein (further including a gene having a nucleotide sequence partly altered) can be used as the immunogen. Further, fragments of the protein may be used. Fragments may be obtained by any method including, but not limited to expressing a fragment of the gene, enzymatic processing of the protein, chemical synthesis, and the like.

In some embodiments, antibodies (e.g., monoclonal antibodies) are humanized. Such humanized antibodies find particular use in the cancer immunotherapies described below. Humanized antibodies are altered in order to make them less immunogenic to humans, e.g., by constructing chimeric antibodies in which a mouse antigen-binding variable domain is coupled to a human constant domain. Humanized antibodies are typically human antibodies in which some CDR residues and possibly some FR residues are substituted by residues from analogous sites in rodent antibodies. Methods for humanizing antibodies are well known in the art and include but are not limited to, those disclosed in U.S. Pat. Nos. 6,054,297, 4,816,567, 6,180,377, 5,871,907, 5,585,089, and 6,180,370, each of which is herein incorporated by reference.

### III. Drug Screening

In some embodiments, the present invention provides drug screening assays (e.g., to screen for drugs that alter Arf and/or Ink4a function). In some embodiments, the screening methods of the present invention utilize Arf and/or Ink4a. For example, in some embodiments, the present invention provides methods of screening for compounds that alter (e.g., decrease) the expression of Arf and/or Ink4a. In other embodiments, candidate compounds are antisense agents (e.g., oligonucleotides) directed against Arf and/or Ink4a. In still further embodiments, candidate compounds are small molecules that inhibit the activity of Arf and/or Ink4a.

In one screening method, candidate compounds are evaluated for their ability to alter (e.g., decrease) Arf and/or Ink4a expression by contacting a compound with a cell expressing Arf and/or Ink4a and then assaying for the effect of the candidate compounds on expression. In some embodiments, the effect of candidate compounds on expression of Arf and/or Ink4a is assayed for by detecting the level of Arf and/or Ink4a mRNA expressed by the cell. mRNA expression can be detected by any suitable method, including but not limited to, those disclosed herein.

In other embodiments, the effect of candidate compounds is assayed by measuring the level of Arf and/or Ink4a polypeptide expression. The level of polypeptide expressed can be measured using any suitable method, including but not limited to, those disclosed herein or by monitoring a phenotype (e.g., prevention of stem cell senescence).

In some preferred embodiments, drug screening assays screen for small molecules that bind and reduce p16Ink4a (or p19Arf) function, or which regulate upstream pathways that promote or inhibit Ink4a (or Arf) expression. Examples of intermediates in upstream pathways that lead to Ink4a expression include, but are not limited to, Jun-b, p38Mapk, or unphysiologically high activation of signaling pathways associated with survival and proliferation (e.g. Ras, PI-3kinase, etc.).

For example, in some embodiments, a gene encoding an indicator is inserted into the Ink4a locus (such as GFP) of stem cells (e.g., neural stem cells or embryonic stem cells) or other cells (e.g., fibroblasts) in culture (e.g., using a vector). A screen is then performed for compounds that modulate GFP expression in the cells. Compounds that increase GFP expression are candidates for inhibiting proliferation or promoting senescence, and find use in cancer therapy. Compounds that decrease GFP expression find use in promoting proliferation or impairing senescence (e.g., as therapies for degenerative diseases or to promote regeneration. In preferred embodiments, screening assays are performed in a high-throughput format.

In some embodiments, in vitro drug screens are performed using purified wild type or dominant active Arf and/or Ink4a and binding partners thereof. Compounds are screened for their ability to interact with Arf and/or Ink4a proteins and inhibit Arf and/or Ink4a function or the interaction of Arf and/or Ink4a with binding partners. In some embodiments, binding partners are immobilized to facilitate separation of complexed from uncomplexed forms of one or both of the proteins, as well as to accommodate automation of the assay. Binding of a test compound to Arf and/or Ink4a proteins is accomplished in any vessel suitable for containing the reactants. Examples of such vessels include microtitre plates, test tubes, and microcentrifuge tubes. In one embodiment, a fusion protein can be provided which adds a domain that allows one or both of the proteins to be bound to a matrix. For example, glutathione-S-transferase/AIP-6 fusion proteins or glutathione-S-transferase/target fusion proteins can be adsorbed onto glutathione sepharose beads (Sigma Chemical; St. Louis, Mo.) or glutathione derivatized microtitre plates, which are then combined with the test compound or the test compound and the non-adsorbed protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly. Alternatively, the complexes can be dissociated from the matrix, and the level of protein binding or activity determined using standard techniques.

Other techniques for immobilizing proteins on matrices can also be used in the screening assays of the invention. For example, downstream Arf and/or Ink4a signaling proteins or other protein known to interact with or modulate signaling by Arf and/or Ink4a can be immobilized utilizing conjugation of biotin and streptavidin. Biotinylated proteins are prepared from biotin-NHS (N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with Arf and/or Ink4a signaling proteins but which do not interfere with binding of the protein to test compounds can be derivatized to the wells of the plate, and unbound protein trapped in the wells by antibody conjugation. Methods for detecting such complexes, in addition to those described above for the GST-immobilized complexes, include immunodetection of complexes using antibodies reactive with Arf and/or Ink4a signaling proteins, as well as enzyme-linked assays that rely on detecting an enzymatic activity associated with Arf and/or Ink4a signaling.

In other embodiments, competitive drug screening assays in which neutralizing antibodies capable of binding Arf and/or Ink4a specifically compete with a test compound for binding to Arf and/or Ink4a are utilized. In this manner, the antibodies can be used to detect the presence of any compound that shares one or more antigenic determinants with Arf and/or Ink4a.

In still further embodiments, genetically altered animals having altered (e.g., inactivated or overexpressed) Arf and/or Ink4a or Bmi-1 genes are utilized in drug screening applications. For example, in some embodiments, compounds are screened for their ability to reduce stem cell senescence in Bmi-1 negative mice.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

IV. Genetically Modified Animals Expressing or Lacking Arf and/or Ink4a

The present invention contemplates the generation of transgenic animals comprising an exogenous Arf and/or Ink4a gene or mutants and variants thereof (e.g., truncations, deletions, insertions, single nucleotide polymorphisms, or heterologous Arf and/or Ink4a genes under control of a promoter that overexpresses the gene)). In other embodiments, the present invention provides transgenic animals with a knock-out of the Arf and/or Ink4a gene. In preferred embodiments, the transgenic animal displays an altered phenotype (e.g., increased or decreased stem cell activity) as compared to wild-type animals. Methods for analyzing the presence or absence of such phenotypes include but are not limited to, those disclosed herein.

The transgenic animals of the present invention find use in drug screens. In some embodiments, test compounds (e.g., a drug that is suspected of being useful to treat a disease characterized by abnormal Arf and/or Ink4a activity) and control compounds (e.g., a placebo) are administered to the transgenic animals and the control animals and the effects evaluated.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter that allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad. Sci. USA 82:6927 [1985]). Transfection is easily and efficiently obtained by culturing the blastomeres on a monolayer of virus-producing cells (Stewart, et al., EMBO J., 6:383 [1987]). Alternatively, infection can be performed at a later stage. Virus or virus-producing cells can be injected into the blastocoele (Jahner et al., Nature 298:623 [1982]). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involve the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40:386 [1995]).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 [1981]; Bradley et al., Nature 309:255 [1984]; Gossler et al., Proc. Acad. Sci. USA 83:9065 [1986]; and Robertson et al., Nature 322:445 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized knock-out gene function or create deletion mutants (e.g., truncation mutants). Methods for homologous recombination are described in U.S. Pat. No. 5,614,396, incorporated herein by reference).

### EXPERIMENTAL

The following examples are provided in order to demonstrate and further illustrate certain preferred embodiments and aspects of the present invention and are not to be construed as limiting the scope thereof.

### Example 1

This Example shows that loss of function of Ink4a and/or Arf rescues self-renewal of Bmi-1−/− neural stem cells in vivo.

### A. Materials and Methods

All mice were bred and maintained in the Unit for Laboratory Animal Medicine at the University of Michigan. The Bmi-1+/−, Arf+/−, and Ink4a/Arf+/− mice were backcrossed at least 8 times onto a C57BL background. Ink4a+/− mice were on an FvB background. All mice were genotyped by PCR using primers. Unless otherwise stated, experiments employed 4 to 8 week old mice.

### Isolation of CNS and PNS Progenitors

Adult SVZ was obtained by coronally sectioning brains in ice-cold Opti-MEM medium (Gibco). The lateral walls of the lateral ventricles were removed, minced, then dissociated for 20 min at 37° C. in 0.025% trypsin/0.5 mM EDTA (Calbiochem, San Diego, Calif.) plus 0.001% DNase1 (Roche, Indianapolis, Ind.). Cells were quenched with staining medium (L15 medium containing 1 mg/ml BSA (Sigma A-3912, St. Louis, Mo.), 10 mM HEPES (pH 7.4), and 1% penicillin/streptomycin (BioWhittaker, Walkersville, Md.)) containing 0.014% soybean trypsin inhibitor (Sigma) and 0.001% DNAse1. After centrifuging, the cells were resuspended in staining medium, triturated, filtered through nylon screen (45 mm, Sefar America, Kansas City, Mo.), counted by hemocytometer, and plated.

For PNS progenitor isolation, adult mouse guts were dissected into ice cold PBS. Outer muscle/plexus layers were peeled free from the underlying epithelium as described (Kruger et al., Neuron 35:657, 2002); then minced, and dissociated for 45 min in 0.025% trypsin/EDTA (Gibco 25300-054, Grand Island, N.Y.) plus 1 mg/ml type 4 collagenase (Worthington) in Ca, Mg-free HBSS at 37° C. with agitation every 5 minutes. The dissociated cells were then quenched in staining medium, resuspended and filtered as described above. Dissociated gut cells were optionally stained with an antibody against p75 (Ab 1554; Chemicon International) as described previously (Bixby et al., Neuron 2002). The analysis and sorting of p75+ cells was performed by a FACS Vantage flow-cytometer (Becton-Dickinson).

### Cell Culture and Self-Renewal Assay

Cells were plated at clonal density on ultra-low binding plates (Corning) to grow neurospheres. Culture medium was based on a 5:3 mixture of DMEM-low glucose: neurobasal medium (Gibco) supplemented with 20 ng/ml human bFGF (R&D Systems, Minneapolis, Minn.), 1% N2 supplement (Gibco), 2% B27 supplement (Gibco), 50 mM 2-mercaptoethanol, and 1% pen/strep (Biowhittaker). CNS cultures also contained 20 ng/ml EGF (R&D Systems) and 10% chick embryo extract (prepared as described (Stemple and Anderson, Cell 71: 973-85, 1992)). PNS cultures contained 15% chick embryo extract, 35 mg/ml (110 nM) retinoic acid (Sigma), and 20 ng/ml IGF1 (R&D Systems). All cultures were maintained at 37° C. in 6% CO₂/balance air.

To measure self-renewal, individual CNS neurospheres were dissociated by trituration then replated at clonal density in non-adherent cultures. Secondary neurospheres were counted 5-10 days later to determine the number of secondary neurospheres formed per primary neurosphere. Individual PNS neurospheres were replated for 48 h into adherent plates to allow the spheres to spread out over the culture dish. The adherent colonies were then treated with trypsin and collagenase (4 parts 0.05% trypsin-EDTA plus 1 part 10 mg/ml collagenase IV) for 5 min at 37° C. followed by trituration. 5,000 dissociated cells were replated per well of a 6 well plate and secondary neurospheres were counted 10 days later.

### Immunocytochemistry of Cultured Cells

CNS neurospheres were tested for multipotency by replating one neurosphere per well into 48-well plates and then culturing adherently for 3 to 5 days prior to triple staining for markers of oligodendrocytes (O4), neurons (TuJ1), and astrocytes (GFAP). Plates were incubated first in anti-04 antibody, and then in donkey anti-mouse IgM secondary antibody conjugated to horse radish peroxidase (Jackson Immunoresearch) followed by nickel diaminobenzidine staining (Shah et al., Cell 85: 331-343, 1996). The cultures were then fixed in acid ethanol (5% glacial acetic acid in 100% ethanol) for 20 min at −20° C. After blocking in PBS containing 4% horse serum, 0.4% BSA, 0.05% sodium azide, and 0.1% Igepal (Sigma), the cultures were stained with anti-Tuj1(1:500, Covance, Princeton, N.J.) and anti-GFAP (1:200, Sigma G-3893) primary antibodies followed by FITC-conjugated goat anti mouse IgG1, and PE-conjugated goat anti mouse IgG2a antibodies (Southern Biotech, Birmingham, Ala.). PNS neurospheres were replated into adherent cultures in which the plates had been coated with poly-D-lysine and human fibronectin as described previously (Bixby et al., Neuron 35: 643-56, 2002). The neurospheres were allowed to differentiate for 7 to 10 days and then fixed in acid ethanol for 20 min at −20° C., washed, blocked, and triply labeled for Peripherin (Chemicon International AB1530), GFAP (Sigma G-3893), and aSMA (Sigma A-2547) as described previously (Shah et al. 1996, supra). Cells were stained for 10 min at room temperature with 10 mg/ml DAPI (Sigma D-8417) to visualize nuclei.

### Immunohistochemistry of Tissue Sections

To quantify SVZ proliferation, mice were injected with 50 mg/kg of bromodeoxyuridine (BrdU) (Sigma), and sacrificed one or two hours after BrdU injection, depending on the experiment. Brains were fixed in 4% paraformaldehyde overnight, then cryoprotected in 15% sucrose, embedded in 7.5% gelatin/15% sucrose, and flash frozen. Twelve μm sections were cut on a Leica cryostat.

For cerebellar histology, 30 um sagital sections were obtained, post-fixed in 10% neutral buffered formalin and stained with hematoxylin and eosin. To study gut neurogenesis, segments of intestine were fixed in 4% paraformaldehyde overnight, then cryoprotected in 30% sucrose, embedded in OCT (Tissue-Tek) and frozen. Twelve μm sections were labeled with the neuronal marker HuC/D, by subjecting the sections to antigen retrieval as recommended by the antibody manufacturer, then pre-blocked for 1 hr at room temperature in modified GSS (PBS containing 5% goat serum, and 0.5% Triton X-100). Primary mouse anti-HuC/D (Molecular Probes, Inc., Eugene, Oreg.) diluted in modified GSS was incubated for 1 h at room temperature, followed by Cy3-conjugated anti-mouse IgG2b (Molecular Probes, Inc., Eugene, Oreg.) for 30 min at room temperature. Slides were counter stained in 2.5 μg/ml DAPI for 10 min at room temperature, and then mounted using ProLong antifade solution (Molecular Probes, Eugene, Oreg.). Only HuC/D+neurons in the myenteric plexus were counted.

### Anti-BrdU Antibody Staining

For detection of BrdU in the CNS tissue sections, DNA was first denatured in 2M HCl for 30 min at room temperature and neutralized with 0.1M Sodium Borate. Sections were preblocked for 1 hr at room temperature in goat serum solution (GSS) (PBS containing 5% goat serum, 1% BSA, and 0.3% Triton X-100 (Sigma)). Primary rat anti-BrdU (1:200, Accurate Chemical, Westbury, N.Y.) diluted in GSS was incubated overnight at 4° C., followed by FITC conjugated anti-rat Fc fragment (Jackson Labs) for 3-4 hrs at room temperature. Slides were counter stained in 2.5 μg/ml DAPI for 10 min at RT, then mounted using ProLong antifade solution (Molecular Probes, Eugene, Oreg.). Numbers of BrdU labeled cells were divided by total DAPI-positive nuclei.

### Western Blots and Quantitative RT-PCR

Cells or tissues were resuspended in ice-cold cell lysis buffer (Cell Signaling Technology) with protease inhibitor cocktail (complete mini tablet, Roche), and incubated for 15-30 minutes on ice. Tissues were briefly homogenized, then all samples were sonicated for 1 minute at 20-30% power in a microson ultrasonic cell disruptor, spun down for 5 minutes at 16,000×g and the supernatant quantified colorimetrically (BioRad protein assay). 50-100 μg of protein per lane were separated in 12% denaturing SDS-PAGE gels and transferred overnight at 4° C. to PVDF membranes (Bio-Rad). The membranes were blocked in Tris buffered saline with 0.1% tween-20 and 5% milk, incubated with primary and secondary antibodies, and washed following standard procedures. Horseradish peroxidase conjugated secondary antibodies were detected by chemiluminescence (ECL Plus; Amersham-Pharmacia). Primary antibodies were mouse monoclonal anti-β-actin (Ab-1; Oncogene), rabbit polyclonal anti-p19ARF (ab80, Abcam), mouse monoclonal anti p21cip1 (Pharmigen) and rabbit polyclonal anti-p16Ink4a (M-156; Santa Cruz Biotechnology).

Quantitative RT-PCR was performed as described previously (Molofsky et al., Nature 425: 962-7, 2003). Primers used to amplify Ink4a were 5′CGAACTCTTTCGGTCGTACCC-3′; SEQ ID NO:1 (sense) and 5′-CGAATCTGCACCGTAGTTGAGC-3′; SEQ ID NO:2 (antisense). These primers amplified an 89 nucleotide product that spanned an intron and that was identical to Ink4a upon sequencing. Primers for cip1 (cdkn1a) were 5-CAGACCAGCCTGACAGATTTC-3; SEQ ID NO:3 (sense) and 5-TCCTGACCCACAGCAGAAGAGG-3: SEQ ID NO:4 (antisense). These primers spanned an intron and yielded a single 109 nucleotide product that was identical to cip1 upon sequencing.

### Primers Used for Mouse Genotyping

Ink4a genotyping was performed by PCR as described (Sharpless et al., Nature 413: 86-91, 2001). Bmi-1 genotyping was performed using the oligonucleotides 5′CGCCGTGCACAGGGTGTCACGTTGCAAGAC-3′; SEQ ID NO:5 and 5′CAAGCCAACCACGGCCTCCAGAAG-3′; SEQ ID NO:6 to detect the gene targeted allele, and the oligonucleotides 5′-AGCAGCAATGACTGTGATGCACTTGAG-3′; SEQ ID NO:7 and 5′GCTCTCCAGCATTCGTCAGTCCATCCC-3′; SEQ ID NO:8 to detect wild-type Bmi-1. Arf genotyping was performed using the oligonucleotides 5′-CGCTGTTCTCCTCTTCCTCATCTC-3′; SEQ ID NO:9 and 5′-CCAGTCATAGCCGAATAGCCTCTC-3′; SEQ ID NO:10 to detect the gene-targeted allele and the oligonucleotides 5′-GCGTACCGCTAAGGGTTCAAAAC-3′; SEQ ID NO: 11 and 5′-TCCTCACAGTGACCAAGAACCTG-3′; SEQ ID NO: 12 to detect wild-type Arf. Ink4a/Arf genotyping (mice targeted to lack the exons common to both Ink4a and Arf) were genotyped using the oligonucleotides 5′-CTGCTCAACTACGGTGCAGATTC-3′; SEQ ID NO:13 and 5′-GCAAATATCGCACGATGTCTTGATG-3′; SEQ ID NO:14 to detect wild-type Ink4a/Arf and the oligonucleotides 5′-CGCTGTTCTCCTCTTCCTCATCTC-3′; SEQ ID NO:15 and 5′-CCAGTCATAGCCGAATAGCCTCTC-3′; SEQ ID NO:16 to detect the gene-targeted allele.

### B. Results

Deletion of Ink4a from Bmi-1−/− Mice Partially Rescues the Frequency of Neural Stem Cells

Although deletion of Ink4a significantly increased the self-renewal of Bmi-1−/− neural stem cells in culture (Molofsky et al., Nature 425: 962-7, 2003), it remained to be determined whether Ink4a deficiency would ameliorate the depletion of Bmi-1−/− stem cells in vivo. Dissociated forebrain subventricular zone (SVZ) cells from 4 to 8 week old mice that were deficient in Bmi-1 and/or Ink4a were cultured to determine the frequency of freshly dissociated cells that could form multipotent neurospheres in culture. Cells were plated at clonal density (2000 cells per well of a 6-well plate) and cultured for 10 days. Neurospheres were counted, then replated to adherent cultures for 3 to 5 days and stained for neurons (TuJ1+), astrocytes (GFAP+), and oligodendrocytes (O4+). As observed previously, a significantly lower percentage of cells from the Bmi-1−/− SVZ formed multipotent neurospheres in culture (FIG. 1A). These Bmi-1−/− neurospheres were significantly smaller than wild-type neurospheres and exhibited defects in proliferation and self renewal as reported previously (Molofsky et al. 2003, supra). Ink4a deficiency significantly increased the percentage of Bmi-1−/− but not Bmi-1+/+ SVZ cells that formed multipotent neurospheres in culture (FIG. 1A). This is consistent with the ability to detect p16Ink4a in Bmi-1−/− but not Bmi-1+/+ SVZ cells in vivo (Molofsky et al. 2003, supra). The magnitude of the increase demonstrated that Ink4a deficiency partially but not completely rescued the frequency of stem cells in the Bmi-1−/− SVZ.

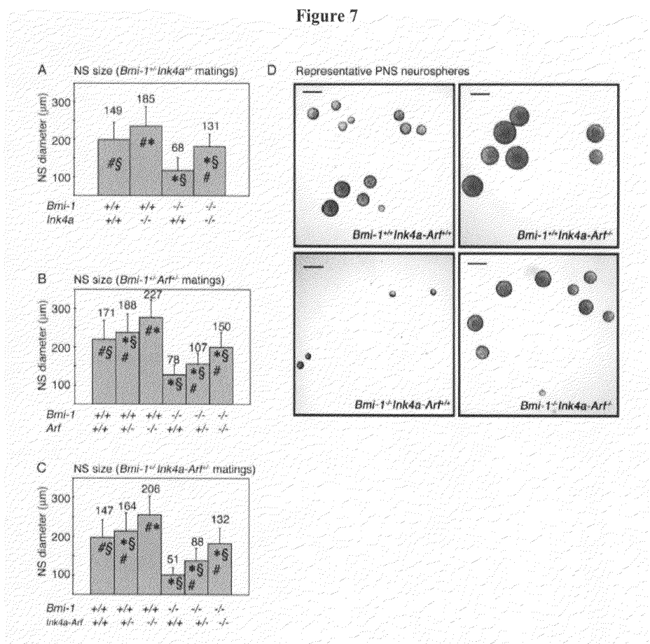

Similar results were obtained from the PNS where NCSCs persist throughout adult life in the outer wall of the gut, in or near the myenteric plexus (Kruger et al., Neuron 35: 657-69, 2002). A significantly lower percentage of cells from the Bmi-1−/− outer gut wall formed multipotent neurospheres in culture (FIG. 1B). These Bmi-1−/− neurospheres were significantly smaller than wild-type neurospheres (FIG. 7) and exhibited defects in proliferation and self-renewal as reported previously (Molofsky et al. 2003, supra). Ink4a deficiency significantly increased the percentage of Bmi-1−/− but not Bmi-1+/+ outer gut wall cells that formed multipotent neurospheres in culture (FIG. 1B). The magnitude of the increase sows that Ink4a deficiency partially but not completely rescued the frequency of stem cells in the Bmi-1−/− gut.

To confirm the partial rescue in neural stem cell frequency in vivo, the ability to prospectively identify uncultured NCSCs by flow-cytometry based on their expression of high levels of the p75 neurotrophin receptor (p75+ cells) (Bixby et al., Neuron 35: 643-56 2002; Kruger et al. 2002, supra) was utilized. In these experiments, 30 to 50% of p75+ cells of all genotypes formed NCSC colonies in culture, a 30 to 300-fold enrichment for NCSCs relative to unfractionated cells (0.1 to 1.4% of unfractionated gut cells, depending on genotype, formed stem cell colonies in culture; FIG. 1B). The frequency of p75+ cells was significantly reduced in the absence of Bmi-1 (FIG. 1C,D). Ink4a deficiency had no effect on the frequency of p75+ cells from Bmi-1+/+ mice, but did significantly increase the frequency of p75+ cells in Bmi-1−/− mice. The magnitude of the increase demonstrated a partial rescue of the Bmi-1−/− stem cell depletion phenotype. The analysis of prospectively identified, uncultured NCSCs showed that part of the mechanism by which Bmi-1 promotes the postnatal maintenance of neural stem cells is by repressing Ink4a.

Ink4a Deficiency Partially Rescues Neural Development, But not Growth or Survival in Bmi-1−/− Mice

In the absence of Bmi-1, there was a significant decline in the rate of proliferation in the adult SVZ (the percentage of cells that incorporated a pulse of BrdU), where neurogenesis occurs in the forebrain (FIG. 2A). Ink4a deficiency had no effect on proliferation in the Bmi-1+/+forebrain but did significantly increase proliferation in the Bmi-1−/− forebrain, suggesting a partial rescue of this phenotype (FIG. 2A). The number of myenteric plexus neurons per cross-section of the adult small intestine was significantly reduced in the absence of Bmi-1 (FIG. 2B). Ink4a deficiency significantly increased the number of neurons per section through the Bmi-1−/− intestine (FIG. 2B). These results demonstrated that Ink4a deficiency partially rescued proliferation/neurogenesis defects in the forebrain and enteric nervous system of Bmi-1−/− mice.

One of the most profound nervous system phenotypes in Bmi-1−/− mice is impaired cerebellum development (van der Lugt et al., Mechanisms of Development 58: 153-164, 1994; Leung et al., Nature 428: 337-41, 2004). Bmi-1−/− mice exhibit morphologically smaller cerebellums, including significantly thinner granular and molecular cell layers, and reduced cell density in the molecular layer (FIG. 2C-E). Ink4a deficiency had no effect on cerebellum development in Bmi-1−/− mice (FIG. 2E), despite the strong upregulation of p16Ink4a in the Bmi-1−/− cerebellum (FIG. 8). This shows that progenitors from some regions of the nervous system are insensitive to p16Ink4a expression.

Although Ink4a deficiency partially rescued certain aspects of stem cell function and neural development in Bmi-1−/− mice, it did not affect the growth of Bmi-1−/− mice, which were significantly smaller than wild-type littermates (FIG. 2F). Ink4a deficiency also did not significantly affect brain mass (FIG. 9) or the survival of Bmi-1−/− mice, which usually died by postnatal day 30 (FIG. 2G), despite being born in nearly expected numbers. These results show that distinct downstream mechanisms mediate the effects of Bmi-1 on neural stem cell maintenance as compared to brain growth, overall mouse growth, and survival.

Arf Deficiency Partially Rescues Neural Stem Cell Self-Renewal and Frequency in Bmi-1−/− Mice

Like p16Ink4a, p19Arf expression increased in the absence of Bmi-1. p19Arf was not detected in the wild-type SVZ or cerebellum, but was detected in these tissues in the absence of Bmi-1 (FIG. 3A). p19Arf was also upregulated in Bmi-1−/− CNS neurospheres, though it was also detected at lower levels in wild-type neurospheres (FIG. 3B). To test the effect of p19Arf on stem cell function, the effect of Arf deficiency on neural stem cell self-renewal and neural stem cell frequency was analyzed. Arf deficiency significantly increased self-renewal within both Bmi-1+/+ and Bmi-1−/− CNS neurospheres (FIG. 3C). Since only about half as much self-renewal was observed within Bmi-1−/−Arf−/− neurospheres as compared to Bmi-1+/+Arf−/− neurospheres, Arf deficiency only partially rescued the self-renewal of Bmi-1−/− neurospheres. Consistent with this, Arf deficiency also significantly increased the size of neurospheres (FIG. 7B), and the percentage of cells within Bmi-1−/− neurospheres that could form secondary neurospheres. Arf deficiency also rescued the depletion of CNS stem cells in Bmi-1−/− mice, significantly increasing the percentage of freshly dissociated SVZ cells from Bmi-1−/− but not Bmi-1+/+ mice that formed multipotent neurospheres in culture (FIG. 3D). This is consistent with the observation of p19Arf expression in the Bmi-1−/− but not Bmi-1+/+ SVZ (FIG. 3A). The data show that increased p19Arf expression in the Bmi-1−/− SVZ leads to reduced stem cell self renewal, and stem cell depletion, and that deletion of Arf partially rescues these defects.

Arf deficiency also partially rescued stem cell self-renewal and stem cell frequency in the PNS. Arf deficiency in the PNS significantly increased the amount of self-renewal within individual Bmi-1+/+ and Bmi-1−/− neurospheres, but self-renewal within Bmi-1−/−Arf−/− neurospheres was only about half that observed in Bmi-1+/+Arf−/− neurospheres (FIG. 3E). Arf deficiency increased the percentage of cells from the outer gut wall of Bmi-1−/− but not Bmi-1+/+ mice that formed multipotent neurospheres in culture (FIG. 3F).

### Arf Deficiency Partially Rescues Neural Development, But not Growth or Survival in Bmi-1−/− Mice

Arf deficiency partially rescued SVZ proliferation and gut neurogenesis in Bmi-1 deficient mice. Arf deficiency significantly increased the rate of proliferation in the Bmi-1−/− but not Bmi-1+/+ SVZ (FIG. 4A). The magnitude of the effect shows a partial rescue of this defect (10.9% of cells in the Bmi-1−/−Arf+/+ SVZ incorporated BrdU as compared to 13.8% in the Bmi-1−/−Arf−/− SVZ and 19.9% in the Bmi-1+/+Arf−/− SVZ; all differences p<0.05; FIG. 4A). Arf deficiency also increased the number of neurons per section through the Bmi-1−/− distal small intestine, though the difference was not statistically significant (FIG. 4B). While Ink4a deficiency had no effect on cerebellum development, Arf deficiency partially rescued cerebellum development in Bmi-1−/− mice. Consistent with the inability to detect p19Arf expression in the cerebellum of Bmi-1+/+ mice (FIG. 3A), Arf deficiency had no effect on cerebellum development in Bmi-1+/+ mice. In contrast, Arf deficiency increased the overall cerebellum size in Bmi-1−/− mice, in addition to significantly increasing the thickness of the granular and molecular layers, and cell density in the molecular layer (FIG. 4C-E). All of these parameters of cerebellum growth remained significantly less than observed in wild-type littermates, indicating a partial rescue (FIG. 4C-E). The observation that Ink4a deficiency had a greater effect on gut neurogenesis, while Arf deficiency had a greater effect on cerebellum development indicates that the relative importance of p16Ink4a and p19Arf differs between progenitor populations and regions of the nervous system. Arf deficiency did not affect the overall growth (FIG. 4F) or survival (FIG. 4G) of Bmi-1−/− mice. Brain mass tended to be higher in adult Bmi-1−/−Arf−/− mice (0.40±0.03 g) as compared to Bmi-1−/−Arf+/+ mice (0.37±0.02 g) but the difference was not significant and both types of mice had smaller brains than wild-type littermates (0.48±0.04 g; p<0.05; FIG. 9B). The rescue of neural stem cell defects without significantly affecting brain mass indicates that the effects of Bmi-1 on stem cells can be uncoupled from its effects on tissue growth.

### Deletion of Both Ink4a and Arf Substantially But Incompletely Rescues Neural Stem Cell Self Renewal and Neural Stem Cell Frequency in Adult Bmi-1 Deficient Mice

Since Ink4a deficiency and Arf deficiency each partially rescued neural stem cell and neural development defects in Bmi-1−/− mice, it was investigated whether deletion of both genes would completely rescue some of these defects. Deletion of Ink4a and Arf (Ink4a-Arf−/−) significantly increased self-renewal within Bmi-1+/+ and Bmi-1−/− CNS neurospheres (FIG. 5A). The rate of self-renewal within Bmi-1−/−Ink4a-Arf−/− neurospheres (390±101 secondary neurospheres/primary neurosphere) and Bmi-1+/+Ink4a-Arf−/− neurospheres (507±193 secondary neurospheres/primary neurosphere; p<0.05) shows that Ink4a-Arf deficiency rescued most but not all of the Bmi-1−/− CNS stem cell self-renewal defect in culture. Ink4a-Arf deficiency also significantly increased the percentage of cells from the Bmi-1−/− but not the Bmi-1+/+ SVZ that formed multipotent neurospheres in culture (FIG. 5B). The percentages of cells from Bmi-1−/−Ink4a-Arf−/− SVZ (2.27±0.81%) and Bmi-1+/+Ink4a-Arf−/− SVZ (3.03±1.59%) that formed multipotent neurospheres shows that Ink4a-Arf deficiency rescued most of the CNS stem cell maintenance defect.

Ink4a-Arf deficiency also substantially, though partially, rescued stem cell self-renewal and maintenance in the PNS. Ink4a-Arf deficiency significantly increased self-renewal within Bmi-1+/+ and Bmi-1−/− PNS neurospheres in culture (FIG. 5C). Comparison of the rate of self renewal within Bmi-1−/−Ink4a-Arf−/− neurospheres (3015±2395 secondary neurospheres/primary neurosphere) and Bmi-1+/+Ink4a-Arf−/− neurospheres (6739±4729 secondary neurospheres/primary neurosphere; p<0.05) shows that Ink4a-Arf deficiency rescued about half of the PNS self-renewal defect. The magnitude of self-renewal observed in these experiments was higher than in previous experiments because the neurospheres were cultured longer before subcloning. Ink4a-Arf deficiency also significantly increased the percentage of cells from the Bmi-1−/− gut that formed multipotent neurospheres in culture (FIG. 5D). The percentages of cells from Bmi-1−/−Ink4a-Arf−/− gut (0.37±0.18%) and Bmi-1+/+Ink4a-Arf−/− gut (0.69±0.43%; p<0.05) that formed multipotent neurospheres show that Ink4a-Arf deficiency rescued about half of the Bmi-1−/− PNS stem cell maintenance defect. These results indicate that increased expression of p16Ink4a and p19Arf in the absence of Bmi-1 are responsible for much but not all of the neural stem cell self-renewal and maintenance defects observed in Bmi-1−/− mice.

### Deletion of Both Ink4a and Arf Partially Rescues Neural Development in Bmi-1 Deficient Mice But not Overall Mouse Growth or Survival

Ink4a-Arf deficiency partially rescued the SVZ proliferation and gut neurogenesis defects observed in Bmi-1 deficient mice. Ink4a-Arf deficiency significantly increased the rate of proliferation in the Bmi-1−/− but not Bmi-1+/+ SVZ in a way that demonstrated a partial rescue (10.8% of cells in the Bmi-1−/−Ink4a-Arf+/+ SVZ incorporated BrdU as compared to 14.7% in the Bmi-1−/− Ink4a-Arf−/− SVZ and 18.4% in the Bmi-1+/+Ink4a-Arf−/− SVZ; all differences p<0.05; FIG. 6A). The magnitude of this rescue was similar to that observed with Ink4a deficiency alone (compare to FIG. 2A) or Arf deficiency alone (compare to FIG. 4A). Ink4a-Arf deficiency also significantly increased the number of neurons per section through the Bmi-1−/− but not Bmi-1+/+ ileum (FIG. 6B). In this case, the data were consistent with a complete rescue (53 neurons/section in the Bmi-1−/−Ink4a-Arf+/+gut as compared to 73 in the Bmi-1−/−Ink4a-Arf−/− gut and 79 in the Bmi-1+/+Ink4a-Arf−/− gut; FIG. 6B). The fact that some neural phenotypes (e.g. gut neurogenesis) were completely rescued by deleting Ink4a and Arf, while other phenotypes (e.g. SVZ proliferation) were only partially rescued indicates that the importance of other pathways downstream of Bmi-1 differs between regions of the nervous system.

Ink4a-Arf deficiency partially but not completely rescued cerebellum development in Bmi-1−/− mice, consistent with a previous study (Jacobs et al., Nature 397: 164-168, 1999a). Ink4a-Arf deficiency had no effect on cerebellum development in Bmi-1+/+ mice, in keeping with the inability to detect p16Ink4a (Molofsky et al. 2003, supra) or p19Arf expression (FIG. 3A) in the Bmi-1+/+ cerebellum. Ink4a-Arf deficiency did increase the overall cerebellum size in Bmi-1−/− mice, including significantly increasing the thickness of the granular and molecular layers, as well as the cell density in the molecular layer (FIG. 6C-E). All of these parameters of cerebellum growth remained significantly less than observed in wild-type littermates, indicating a partial rescue (FIG. 6C-E). The magnitude of the rescue in Bmi-1−/− cerebellum development from Ink4a-Arf deletion was similar to that observed from deleting Arf alone (compare FIG. 6CE to FIG. 4C-E), consistent with the lack of effect of Ink4a deficiency (FIG. 2C-E).

Like Ink4a deficiency and Arf deficiency, Ink4a-Arf deficiency also did not affect overall growth (FIG. 6F) or survival (FIG. 6G). Brain mass was significantly higher in adult Bmi-1−/−Ink4a-Arf−/− mice (0.39±0.02 g) as compared to Bmi-1−/−Ink4a-Arf+/+ mice (0.36±0.01 g; p<0.05), though still significantly reduced relative to wild-type littermates (0.49±0.02 g; p<0.05; FIG. 9C). This is in keeping with the increased size of the cerebellum in Bmi-1-Ink4a-Arf−/− mice as compared to Bmi-1−/−Ink4a-Arf+/+ mice (FIG. 6C-E) but could reflect effects on the size of other brain regions as well. These results demonstrate that other pathways account for the effect of Bmi-1 on overall mouse growth and survival.

Neural Stem Cell Frequency and Self-Renewal Decline with Age in Normal Mice

Since Bmi-1 promotes the self-renewal of neural stem cells by repressing senescence pathways, it was investigated what happens as these stem cells age. Forebrain stem cells decline in frequency and mitotic activity with age (Maslov et al., J Neurosci 24: 1726-33, 2004), but the effect of age on self-renewal potential has not been studied and the effects of aging on PNS stem cells are unknown. A two-fold reduction in the frequency of CNS stem cells and an approximately three-fold reduction in the rate of proliferation in the SVZ was found between P60 and 2 years of age. This decline in stem cell frequency was associated with an approximately two-fold reduction in self-renewal potential. PNS stem cells in the outer wall of the gut declined in frequency around 10-fold, and in self-renewal potential about two-fold from young adulthood to old age. Consistent with this, neurospheres cultured from the guts of old animals were qualitatively smaller than those cultured from young adult animals.

Increased p16Ink4a Expression in Aging Neural Stem Cells

Since increased p16Ink4a and p19Arf expression reduce stem cell frequency and self-renewal potential in the CNS and PNS (FIG. 1, 3), and reduce mitotic activity in the SVZ (FIG. 2, 4), it was investigated whether the age-related changes observed in wild-type mice were associated with increases in p16Ink4a or p19Arf expression. p16Ink4a and p19Arf expression increase with age in some mouse tissues (Zindy et al., Oncogene 15: 203-211, 1997; Krishnamurthy et al., J Clin Invest 114: 1299-307, 2004), though these studies did not examine stem cells. No age-related increase was detected in p19Arf expression in neural progenitors at the RNA or protein levels. In contrast, Ink4a (RNA) expression increased substantially with age in both uncultured SVZ cells and uncultured p75+NCSCs from the gut wall. An increase in p16Ink4a protein expression was observed in the gut wall. No p16Ink4a protein expression was detected in the SVZ.

Subtle reductions in Bmi-1 expression are functionally important as even the 2-fold reduction in Bmi-1 expression in Bmi-1−/+ mice leads to increased p16Ink4a expression. These data indicate that age-related declines in neural stem cell frequency and self renewal potential are associated with increased p16Ink4a expression, at least in gut NCSCs, despite ongoing Bmi-1 expression. This age-related increase in p16Ink4a expression was less dramatic than what is observed in response to Bmi-1 deficiency (Molofsky et al. 2003, supra), consistent with the less dramatic age-related decline in stem cell frequency and self-renewal as compared to what is observed in response to Bmi-1 deficiency.

### Example 2

This Example shows that loss of function of Ink4a partially rescues the decline in forebrain progenitor activity that is normally observed with age. This demonstrates that part of the mechanism responsible for the decline in progenitor activity and regenerative capacity in aging tissues is the age-related increase in p16Ink4a expression.

### A. Methods

Ink4a+/− mice were backcrossed at least six times onto a C57BL background. The design and validation of Ink4a BAC transgenic mice is described elsewhere. In brief, eleven C57B1/6×C3H transgenic lines were generated through pronuclear injection of a linearized 65 kb genomic 129SvEv BAC clone containing exon 1 alpha, exon 2 and exon 3 (of Ink4a), but not exon 1beta (of Arf) or any exons of p15Ink4b. Lines were screened by Southern blotting using an exon 1 apha probe, and two single copy integrant lines chosen for further characterization. The experimental cohort of transgenic mice and littermate controls was generated after backcrossing two generations to C57B1/6 and was housed and aged in a barrier facility on a standard diet.

### Isolation of CNS Progenitors

Adult SVZ was obtained by coronally sectioning brains in ice-cold Opti-MEM medium (Gibco). The lateral walls of the lateral ventricles were removed, minced, then dissociated for 20 min at 37° C. in 0.025% trypsin/0.5 mM EDTA (Calbiochem, San Diego, Calif.) plus 0.001% DNase1 (Roche, Indianapolis, Ind.). Cells were quenched with staining medium (L15 medium containing 1 mg/ml BSA (Sigma A-3912, St. Louis, Mo.), 10 mM HEPES (pH 7.4), and 1% penicillin/streptomycin (BioWhittaker, Walkersville, Md.)) containing 0.014% soybean trypsin inhibitor (Sigma) and 0.001% DNAse1. After centrifuging, the cells were resuspended in staining medium, triturated, filtered through nylon screen (45 mm, Sefar America, Kansas City, Mo.), counted by hemocytometer, and plated.

### Cell Culture and Self-Renewal Assay

Cells were plated at clonal density on ultra-low binding plates (Corning) to grow neurospheres. Culture medium was based on a 5:3 mixture of DMEM-low glucose: neurobasal medium (Gibco) supplemented with 20 ng/ml human bFGF (R&D Systems, Minneapolis, Minn.), 20 ng/ml EGF (R&D Systems), 1% N2 supplement (Gibco), 2% B27 supplement (Gibco), 50 mM 2-mercaptoethanol, and 1% pen/strep (Biowhittaker), and 10% chick embryo extract (prepared as described; Stemple and Anderson, Cell 71, 973-85 (1992)). Cultures were maintained at 37° C. in 6% CO₂/balance air. To measure self-renewal, individual CNS neurospheres were dissociated by trituration then replated at clonal density in non-adherent cultures. Secondary neurospheres were counted 5-10 days later to determine the number of secondary neurospheres formed per primary neurosphere.

### Immunocytochemistry

CNS neurospheres were tested for multipotency by replating one neurosphere per well into 48-well plates and then culturing adherently for 3 to 5 days prior to triple staining for markers of oligodendrocytes (O4), neurons (TuJ1), and astrocytes (GFAP). To quantify SVZ proliferation, mice were injected with 50 mg/kg of bromodeoxyuridine (BrdU) (Sigma), and sacrificed two hours after BrdU injection, depending on the experiment.

Brains were fixed in 4% paraformaldehyde overnight, then cryoprotected in 15% sucrose, embedded in 7.5% gelatin/15% sucrose, and flash frozen. Twelve μm sections were cut on a Leica cryostat. Observers were blind with respect to sample identity (genotype) when counting the frequency of BrdU+ cells.

To stain neural progenitor colonies for makers of neurons (TuJ1), astrocytes (GFAP), and oligodendrocytes (O4), plates were incubated first in anti-04 antibody, and then in donkey antimouse IgM secondary antibody conjugated to horse radish peroxidase (Jackson Immunoresearch) followed by nickel diaminobenzidine staining. The cultures were then fixed in acid ethanol (5% glacial acetic acid in 100% ethanol) for 20 min at −20° C. After blocking in PBS containing 4% horse serum, 0.4% BSA, 0.05% sodium azide, and 0.1% Igepal (Sigma), the cultures were stained with anti-Tuj1(1:500, Covance, Princeton, N.J.) and anti-GFAP (1:200, Sigma G-3893) primary antibodies followed by FITC-conjugated goat anti mouse IgG1, and PE conjugated goat anti mouse IgG2a antibodies (Southern Biotech, Birmingham, Ala.). Cells were stained for 10 min at room temperature with 10 mg/ml DAPI (Sigma D-8417) to visualize nuclei.

For detection of BrdU in the CNS tissue sections, DNA was first denatured in 2M HCl for 30 min at room temperature and neutralized with 0.1M Sodium Borate. Sections were preblocked for 1 hr at room temperature in goat serum solution (GSS)(PBS containing 5% goat serum, 1% BSA, and 0.3% Triton X-100 (Sigma)). Primary rat anti-BrdU (1:500, Accurate Chemical, Westbury, N.Y.) diluted in GSS was incubated overnight at 4° C., followed by FITC conjugated anti-rat Fc fragment (Jackson Labs) for 3-4 hrs at room temperature. Slides were counter stained in 2.5 μg/ml DAPI for 10 min at RT, then mounted using ProLong antifade solution (Molecular Probes, Eugene, Oreg.). Numbers of BrdU labeled cells were divided by total DAPI-positive nuclei.

### Western Blots and Quantitative RT-PCR

Cells or tissues were resuspended in ice-cold cell lysis buffer (Cell Signaling Technology) with protease inhibitor cocktail (complete mini tablet, Roche), and incubated for 15-30 minutes on ice. Tissues were briefly homogenized, then all samples were sonicated for 1 minute at 20-30% power in a microson ultrasonic cell disruptor, spun down for 5 minutes at 16,000 g and the supernatant quantified colorimetrically (BioRad protein assay). 50-100 μg of protein per lane were separated in 12% denaturing SDS-PAGE gels and transferred overnight at 4° C. to PVDF membranes (Bio-Rad). The membranes were blocked in Tris buffered saline with 0.1% tween-20 and 5% milk, incubated with primary and secondary antibodies, and washed following standard procedures. Horseradish peroxidase conjugated secondary antibodies were detected by chemiluminescence (ECL Plus; Amersham-Pharmacia). Primary antibodies were mouse monoclonal anti-β-actin (Ab-1; Oncogene), mouse monoclonal anti-Bmi-1 (Upstate biotechnologies), and rabbit polyclonal anti-p16Ink4a (M-156; Santa Cruz Biotechnology).

Quantitative RT-PCR was performed as described previously (Molofsky, 2003, supra). Primers used to amplify Ink4a were 5′CGAACTCTTTCGGTCGTACCC-3′; SEQ ID NO:17 (sense) and 5′-CGAATCTGCACCGTAGTTGAGC-3′; SEQ ID NO:18 (antisense). These primers amplified an 89 nucleotide product that spanned an intron and that was identical to Ink4a upon sequencing.

### Primers Used for Mouse Genotyping

Ink4a genotyping was performed by PCR as described (Sharpless et al., Nature 413, 86-91 (2001)). Ink4a BAC transgenic mice were genotyped by PCR using the oligonucleotides 5′-CGATCCTCCCGAATTGACTA-3′; SEQ ID NO:19 and 5′-GCACATTCTGCACTACAACTCC-3′; SEQ ID NO:20 to amplify the transgene.

### BrdU Incorporation/Proliferation Assays

To quantitate SVZ proliferation, mice were injected intraperitoneally with 50 mg/kg of bromodeoxyuridine (BrdU)(Sigma), and sacrificed two hours after BrdU injection. To quantitate proliferation in the dentate gyrus, mice received a single injection of 50 mg/kg BrdU, followed by 1 mg/ml BrdU in their drinking water for 8 days before being sacrificed and analyzed. To quantitate neurogenesis in the dentate gyrus and the olfactory bulb and BrdU retention in the SVZ, mice were treated with BrdU for 8 days. The animals were then taken off of BrdU for 4 weeks before the animals were sacrificed. The brains were dissected, fixed in 4% paraformaldehyde overnight, then cryoprotected in 15% sucrose, embedded in 7.5% gelatin/15% sucrose, and flash frozen. 12 μm sections were cut on a Leica cryostat.

For detection of BrdU in the tissue sections, DNA was denatured in 2M HCl for 30 min at room temperature (RT) and neutralized with 0.1M Sodium Borate. Sections were pre-blocked for 1 hr at RT in goat serum solution (GSS) (PBS containing 5% goat serum, 1% BSA, and 0.3% Triton X-100 (Sigma)). Primary rat anti-BrdU (1:500, Accurate Chemical, Westbury, N.Y.) diluted in GSS was incubated overnight at 4° C., followed by FITC-conjugated anti-rat Fc fragment (Jackson Labs) for 3-4 hrs at RT. Slides were counter stained in 2.5 μg/ml DAPI for 10 min at RT, then mounted using ProLong antifade solution (Molecular Probes, Eugene, Oreg.). Numbers of BrdU labeled cells were divided by total DAPI+ or NeuN+ nuclei.

To ensure that BrdU incorporation results were not skewed by dividing endothelial or hematopoietic cells, SVZ sections were double layered with antibodies against BrdU and either anti-CD45 (to identify blood cells) or anti-VE-cadherin (to identify endothelial cells). There was no detection of BrdU+hematopoietic or endothelial cells.

### Confocal Analysis of Neurogenesis in the Olfactory Bulb

A Zeiss LSM 510 confocal laser-scanning microscope was used to obtain 25-30 random fields of view throughout all regions of one entire olfactory bulb of each mouse with a 40× or 63× objective lens. For each image, 1 μm thick optical sections were scanned with 3 different lasers through a 12 μm sagital tissue section creating a Z-series stack with 3 distinct channels of florescence. A UV enterprise laser was used to detect the DAPI signal labeling all nuclei. An argon laser detected FITC (BrdU), and a HeNe laser detected Cy3 (NeuN). Channels were merged together to determine whether DAPI, BrdU, and NeuN signals co-labeled at every 1 um slice of the Z-series. Three dimensional projections were made using LSM 510 software.

### B. Results

Ink4a gene expression increases with age in a variety of tissues (Nielsen et al., Lab Invest 79, 1137-43 (1999); Krishnamurthy et al., J Clin Invest 114, 1299-307 (2004); Zindy et al., Oncogene 15, 203-211 (1997)). Induction of Ink4a expression can cause the senescence of a variety of cell types in culture (Lowe et al., Current Opinion in Genetics & Development 13, 77-83 (2003); Sherr, Nature Reviews Molecular Cell Biology 2, 731-737 (2001); Sharpless et al., Oncogene 23, 379-85 (2004)). Various measures of progenitor frequency and function were examined in the forebrain lateral ventricle subventricular zone (SVZ) of aging wild-type, Ink4a-deficient, and Ink4a BAC transgenic mice. The SVZ contains a mixed population of stem cells and other progenitors that engage in neurogenesis throughout adult life (Maslov et al., supra; Enwere et al., J Neurosci 24, 8354-65 (2004)).

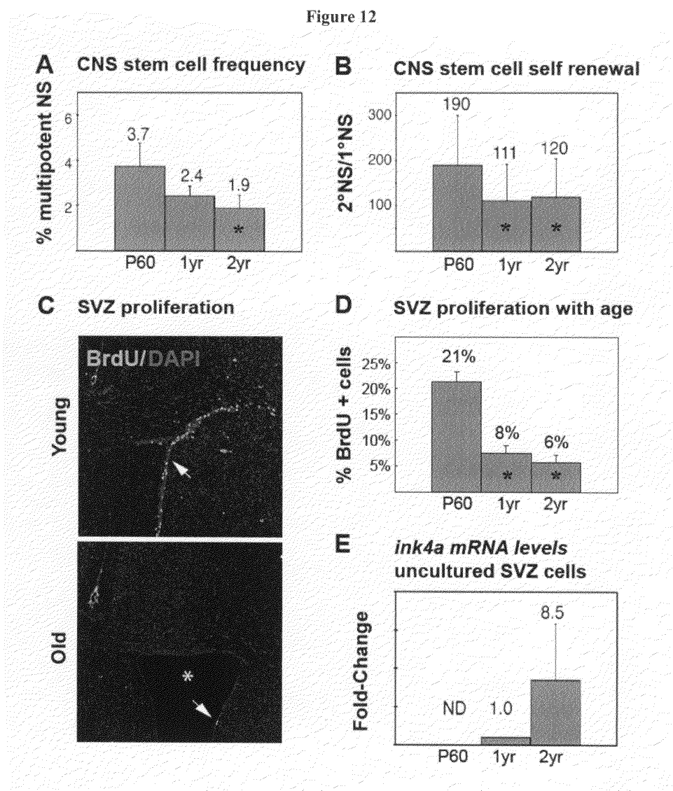

Various measures of progenitor function were compared in the SVZ of 60 day old (P60), 1 year old, and 2 year old mice to determine the effect of aging on CNS progenitor function. A two-fold reduction was found between P60 and 2 years of age in the frequency of SVZ cells that formed multipotent neurospheres in culture (FIG. 12A; *, p<0.05). This decline was also associated with an approximately two-fold reduction in the self-renewal potential of these multipotent neurospheres (FIG. 12B; *, p<0.05). In vivo, an approximately three-fold reduction was observed in the rate of proliferation in the SVZ between P60 and 2 years of age (FIG. 12C, D; *, p<0.05). These data show that stem cell frequency and self renewal potential decline with age in the SVZ and that the overall proliferation rate in the SVZ in vivo also declines.

Adult Bmi-1-deficient mice also exhibit reduced stem cell frequency and self-renewal potential, as well as reduced progenitor proliferation in the SVZ (Molofsky, 2003, supra; Zencak et al., J Neurosci 25, 5774-83 (2005)). These effects are largely caused by increased Ink4a and Arf expression in the absence of Bmi-1 (Molofsky, 2003, supra; Molofsky et al., Genes & Development 19, 1432-1437 (2005); Bruggeman et al., Genes & Development 19, 1438-1443 (2005)). It was investigated whether the age-related changes observed in wild-type mice (FIG. 12) were associated with increased Ink4a or Arf expression in neural progenitors. Ink4a and Arf expression increased with age in some tissues (Krishnamurthy et al., supra, Zindy et al., supra). No age-related increase in Arf expression was detected in uncultured SVZ cells at the RNA or protein levels. Ink4a expression increased substantially with age in uncultured SVZ cells (FIG. 12E). Ink4a expression was not detectable by PCR in the SVZ of 60 day old mice, but became detectable by 1 year of age, and went on to increase a further 8.5 fold by 2 years of age.

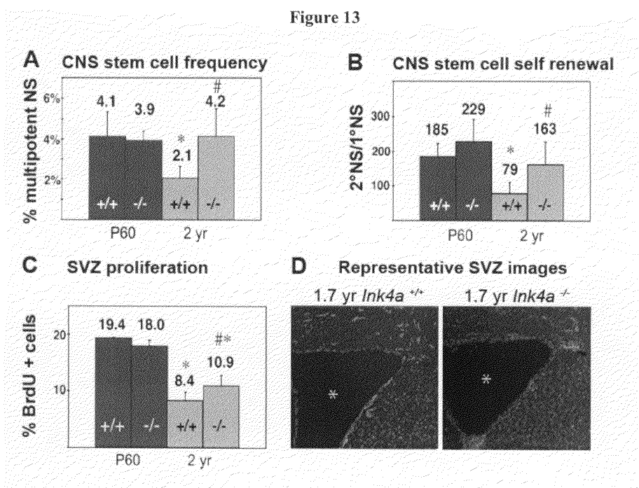

To test the function of Ink4a in aging neural progenitors, young (P60) and old (2 year old) Ink4a-deficient mice as well as littermate controls were examined. Deletion of Ink4a did not significantly affect the frequency of SVZ cells from young mice that formed multipotent neurospheres in culture (FIG. 13A), or the self-renewal potential of these cells upon subcloning (FIG. 13B). This is consistent with the failure to detect Ink4a expression in SVZ cells from young mice in vivo. Ink4a deficiency significantly increased the frequency of SVZ cells from old mice that formed multipotent neurospheres in culture (FIG. 13A; #, p<0.05 relative to old wild-type), as well as the self-renewal potential of these cells (FIG. 13B; #, p<0.05 relative to old wild-type). Consistent with this, the rate of proliferation among SVZ cells in vivo was also significantly increased by Ink4a deficiency in old but not young mice (FIG. 13C; #, p<0.05 relative to old wild-type). Nonetheless, the percentage of proliferating cells in old Ink4a-deficient mice was still significantly less than observed in normal or Ink4a-deficient young mice (FIG. 13C; *, p<0.05 relative to young mice). Like old wild-type mice, old Ink4a-deficient mice also exhibited an enlargement of the lateral ventricle due to cortical atrophy (FIG. 13D). These observations indicate that Ink4a deficiency only partially rescued the age-related declines in progenitor activity in the old SVZ and did not prevent cortical atrophy.

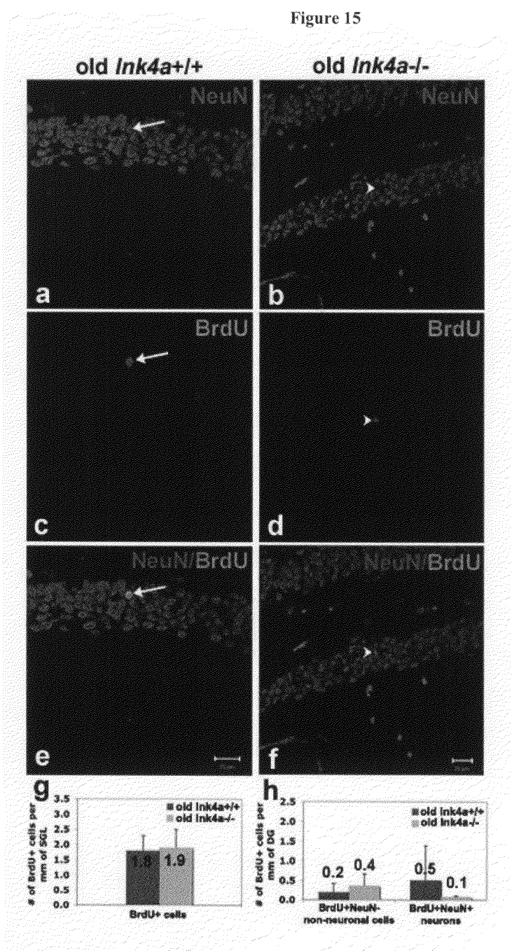

SVZ progenitors form neuroblasts throughout life that migrate into the olfactory bulb and differentiate into neurons (Enwere et al., J Neurosci 24, 8354-65 (2004); Doetsch et al., Cell 97, 703-716 (1999)). To test whether the increase in Ink4a expression within the SVZ also affects neurogenesis, the effects of age and Ink4a-deficiency on the generation of new neurons in the olfactory bulb was examined. BrdU was administered to mice for 8 days to mark dividing progenitors, followed by a four week chase period with no BrdU (during which neurons could migrate and differentiate). The mice were then sacrificed to analyze sections through the olfactory bulb by confocal microscopy. Ink4a-deficiency had no effect on neurogenesis in young mice, but neurogenesis significantly decreased with age, and Ink4a-deficiency significantly increased the frequency of newborn olfactory bulb neurons in 15 to 19 month old mice (FIG. 15i). Ink4a-deficiency had no effect on the frequency of non-neuronal cells within the olfactory bulb (FIG. 15j). Ink4a deficiency thus partially rescued the age-related decline in neurogenesis in the olfactory bulb in addition to partially rescuing the decline in progenitor function in the SVZ.

To test whether these effects of Ink4a occurred throughout the CNS, the effect of Ink4a deficiency on progenitor activity and neurogenesis in the dentate gyrus (van Praag et al., Nature 415, 1030-4. (2002)) was examined. The rates of progenitor proliferation and neurogenesis in the dentate gyrus decline dramatically with age (Kuhn et al., Journal of Neuroscience 16, 2027-2033 (1996)). To test whether this is affected by Ink4a, BrdU was administered for 8 days to mark proliferation in the subgranular layer, followed by a four week chase period without BrdU to examine neurogenesis in the granular layer. Ink4a-deficiency did not significantly affect the rate of proliferation among progenitors in the subgranular layer, or the frequency of BrdU+NeuN+ newborn neurons or BrdU+NeuN− non-neuronal cells in the granular layer of the dentate gyrus (FIG. 15). Thus while Ink4a deficiency consistently increased all measures of progenitor activity and neurogenesis in the aging subventricular zone/olfactory bulb, it did not detectably affect proliferation or neurogenesis in the dentate gyrus.

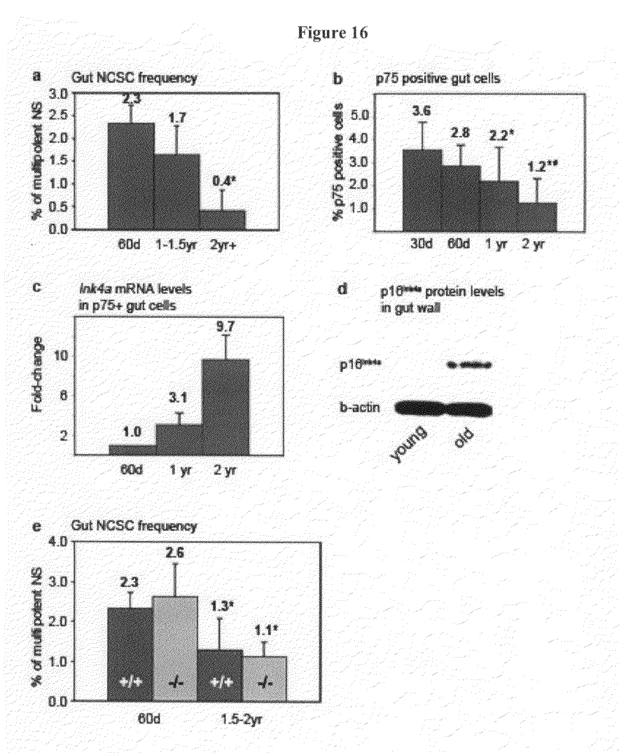

The effect of age and Ink4a deficiency on the neural crest stem cells that persist throughout adult life in the enteric nervous system (in the gut wall) (Kruger et al., Neuron 35, 657-69. (2002); Molofsky et al., Genes & Development 19, 1432-1437 (2005); Molofsky et al., Nature 425, 962-7 (2003)) was also examined. The frequency of these neural crest stem cells declined with age and Ink4a and p16Ink4a expression increased with age in cell populations enriched for neural crest stem cells (FIG. 16). However, Ink4a deficiency had no effect on neural crest stem cell frequency in young or old mice. The present invention is not limited to a particular mechanism. Indeed, an understanding of the mechanism is not necessary to practice the present invention. Nonetheless, it is contemplated that these results indicate that while the age-related increase in Ink4a expression causes a decline in SVZ progenitor function and neurogenesis, other mechanisms are more important in the aging of stem and progenitor cells in the hippocampus and peripheral nervous system.

The present invention is not limited to a particular mechanism. Indeed, an understanding of the mechanism is not necessary to practice the present invention. Nonetheless, it is contemplated that Ink4a expression is developmentally programmed to increase with age to counter the increasing incidence of cancer that is observed with age in the nervous system and other tissues. Alternatively, increased Ink4a expression may reflect the induction of senescence pathways in aging cells in response to damage that accumulates from oxidative metabolism or other mutagens. Although Bmi-1 is an important repressor of Ink4a (Molofsky et al., 2003, supra; Molofsky et al., 2005, supra; Jacobs et al., Nature 397, 164-168 (1999)), Bmi-1 did not show any clear age-related changes in expression at the RNA or protein levels in SVZ cells (FIG. 15).

Moderately increased expression of p16Ink4a and p19Arf in transgenic mice did not detectably accelerate gross measures of aging or change lifespan (Matheu et al., Genes Dev 18, 2736-46 (2004)). The present example did not examine neural progenitor function. The magnitude of the increase in p16Ink4a and p19Arf expression in those transgenic mice was smaller than the increase observed in old wild-type mice or in the Ink4a transgenic mice used in this study. It is contemplated that the levels of Ink4a that are required to promote age-related declines in progenitor function are qualitatively higher than the levels that are required to inhibit cancer. The data indicating that Ink4a deficiency reduces the age-related decline in the frequency and function of neural progenitors in old mice demonstrates that the elevated levels of Ink4a in old mice do reduce progenitor function, despite the lack of any effect of Ink4a deficiency on neural progenitor function in young mice. Although Ink4a regulates age-related declines in stem/progenitor cell function, how this relates to overall aging or lifespan remains unclear. It is contemplated that declining stem/progenitor cell function is a major cause of the decline in regenerative capacity and the increase in degenerative disease that is observed in aging tissues. Increases in the death or dysfunction of mature cells in aging tissues also contribute to age-related morbidity. The results of this example show that adult stem/progenitor cell function is regulated by a balance between the repression (through young adulthood) and activation (in old adults) of Ink4a (Molofsky et al., 2003, supra; Molofsky et al., 2005, supra). It is contemplated that other pathways beyond Bmi-1 also regulate stem cells by influencing this balance (Ito et al., Nature 431, 997-1002 (2004); Sun et al., Genes Dev 18, 1035-46 (2004)). This reflects the physiological consequences of mechanisms that evolved to balance the need for regenerative capacity in tissues while guarding against neoplastic proliferation that becomes an increasing risk with age. The influence of Ink4a thus reduces cancer incidence (Lowe et al., supra; Sharpless et al., Nature 413, 86-91 (2001)) but also contributes to aging by reducing progenitor function in aging tissues.

In conclusion, this example demonstrates that an age-related increase in p16Ink4a expression is at least partially responsible for the decline in progenitor activity and regenerative capacity in aging tissues.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A method of promoting stem cell function comprising inhibiting the function of a gene selected from the group consisting of Arf and Ink4a in a stem cell.

2. The method of claim 1, wherein said stem cell is in vitro.

3. The method of claim 1, wherein said stem cell is ex vivo.

4. The method of claim 1, wherein said stem cell is in vivo.

5. The method of claim 1, wherein said inhibiting the function of a gene comprises contacting said stem cell with an siRNA complementary to said gene.

6. The method of claim 1, wherein said inhibiting the function of a gene comprises contacting said stem cell with a small molecule that reduces Ink4a expression or p16Ink4a function.

7. The method of claim 1, wherein said inhibiting the function of a gene comprises inhibiting both Ink4a and Arf function.

8. The method of claim 1, wherein said stem cell is in a subject having symptoms of a degenerative disease.

9. The method of claim 8, wherein said inhibiting the function of a gene reduces symptoms of said degenerative disease.

10. The method of claim 1, wherein said stem cell is a neural stem cell.

11. The method of claim 1, wherein said stem cell is in a subject having symptoms of a neurodegenerative disease.

12. The method of claim 1, wherein said stem cell lacks a functional Bmi gene.

13. A method of screening compounds, comprising contacting a neural stem cell that expresses a gene selected from the group consisting of Arf and Ink4a with a test compound; and comparing the level of proliferation in said neural stem cell in the presence of said test compound with said level in the absence of said test compound.

14. The method of claim 13, wherein said test compound is an antisense oligonucleotide complementary to said gene.

15. The method of claim 13, wherein said test compound is an siRNA complementary to said gene.

16. The method of claim 13, wherein said test compound is a small molecule that inhibits the function of said gene.

17. The method of claim 13, wherein said test compound inhibits Ink4a activity.

18. The method of claim 13, wherein said cell is in a non-human animal.

19. The method of claim 18, wherein said non-human animal is lacking a functional Bmi-1 gene.

20. A method of screening compounds, comprising contacting a stem cell that expresses a gene selected from the group consisting of Arf and Ink4a with a test compound; and comparing the level of expression of said gene in the presence of said test compound with said level in the absence of said test compound.
